(19) United States
(12) Patent Application Publication
(10) Pub. No.: US 2022/0105062 A1
(43) Pub. Date: Apr. 7, 2022

(54) TRANSDERMAL PENETRANT FORMULATIONS

(71) Applicant: Dyve Biosciences, Inc., Thousand Oaks, CA (US)

(72) Inventors: Nathan FITZSIMMONS, Newbury Park, CA (US); Ryan BEAL, Thousand Oaks, CA (US); Brandon SAND, Camarillo, CA (US)

(21) Appl. No.: 17/554,449

(22) Filed: Dec. 17, 2021

Related U.S. Application Data
(63) Continuation of application No. PCT/US2020/038558, filed on Jun. 18, 2020.
(60) Provisional application No. 62/863,242, filed on Jun. 18, 2019.

(51) Int. Cl. A61K 31/195; A61K 9/00; A61K 47/24; A61K 47/26; A61K 47/12; A61K 47/10; A61K 31/045; A61P 35/00

(52) U.S. Cl. CPC A61K 31/195; A61K 9/0014; A61K 47/24; A61P 35/00; A61K 47/12; A61K 47/10; A61K 31/045; A61K 47/26

(57) ABSTRACT

Disclosed herein is a transdermal delivery formulation for transdermal delivery of an active agent through the skin, nail or hair follicle of a subject, wherein the formulation comprises a) a transdermal delivery formulation in an amount less than about 60% w/w, comprising i. one or more phosphatides, ii. glucose, and iii. one or more fatty acids; and b) water in an amount less than about 50% w/w.

### CROSS-REFERENCE TO RELATED APPLICATIONS

This application is a Continuation application of PCT/US2020/038558, filed Jun. 18, 2020, which claims the benefit of U.S. Provisional Application No. 62/863,242, filed Jun. 18, 2019. Each of the priority applications are incorporated herein by reference in its entirety.

### BACKGROUND

Lecithin organogel is a common component of transdermal penetrants. Lecithin is used as it provides several advantageous effects on the ability of an active ingredient to penetrate an individual's skin. Additionally, there is no one lecithin organogel formulation that can be used, but several, including soy lecithin. Each lecithin formulation is comprised of a number of components, including phosphatides and fatty acids. Moreover, while lecithin organogel has advantages, there ae also disadvantages to using lecithin organogel. For instance, there are some minor components that can have negative effects on the stability of formulations with lecithin organogel. These include carotenoids (i.e., Lutein), which can result in the color of the lecithin falling within a spectrum from brown to yellow, with the result that the final color observed by a consumer and/or patient can be undesirable. Furthermore, soy lecithin organogel possesses a notable smell that consumers and/or patients may find to be unpleasant. Transdermal preparations containing lecithin organogel are also known to have the potential to separate into different fractions over time, resulting in an unpalatable look. Lecithin organogel can further result in a gritty feel to the cream as it is rubbed on an individual. Finally, as soy lecithin is derived from a natural product it tends to lend itself to a higher range of variability in color, feel, and look from batch to batch, which is undesirable for commercial applications.

Therefore, there is a need for an improved transdermal formulation that addresses the aforementioned drawbacks and achieves improved penetration, color, smell and stability as compared to formulations containing lecithin organogel.

Aspects of the present invention fulfill these needs and provide further related advantages as described in the following summary.

### SUMMARY

Aspects of the present disclosure teach certain benefits in construction and use which give rise to the exemplary advantages described below.

The present disclosure solves the problems described above by providing therapeutic agent formulations with improved penetration. In at least one embodiment, disclosed herein are transdermal penetrant formulations that do not contain lecithin organogel.

In one aspect, disclosed herein is a transdermal delivery formulation of an active agent through the skin, nail or hair follicle of a subject, wherein the formulation comprises a) a transdermal delivery formulation in an amount less than about 60% w/w, comprising i. one or more phosphatides and ii. one or more fatty acids; and b) water in an amount less than about 50% w/w.

In another aspect, disclosed herein is a transdermal delivery formulation of an active agent through the skin, nail or hair follicle of a subject, wherein the formulation comprises a) a transdermal delivery formulation in an amount less than about 60% w/w, comprising i. one or more phosphatides and ii. one or more fatty acids; and b) water in an amount less than about 50% w/w, and an active agent.

In another aspect, disclosed herein is a method to effect transdermal delivery of an active ingredient comprising applying to the skin, nails or hair follicles of a subject an effective amount of a transdermal delivery formulation of an active agent through the skin, nail or hair follicle of a subject, wherein the formulation comprises a) a transdermal delivery formulation in an amount less than about 60% w/w, comprising i. one or more phosphatides and ii. one or more fatty acids; and b) water in an amount less than about 50% w/w, and an active agent.

Other features and advantages of aspects of the present invention will become apparent from the following more detailed description, taken in conjunction with the accompanying drawings, which illustrate, by way of example, the principles of aspects of the invention.

### DETAILED DESCRIPTION

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

In an embodiment, a “pharmaceutical composition” is intended to include, without limitation, the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers, without limitation, to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will, without limitation, vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effected may, without necessarily being therapeutic, also be a cosmetic effect, in particular for treatment for disorders of the skin described herein.

In an embodiment, a “subject” of diagnosis or treatment is, without limitation, a prokaryotic or a eukaryotic cell, a tissue culture, a tissue or an animal, e.g. a mammal, including a human. Non-human animals subject to diagnosis or treatment include, for example, without limitation, a simian, a murine, a canine, a leporid, such as a rabbit, livestock, sport animals, and pets.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

For purposes herein, a formulation, a formulation for transdermal delivery and a transdermal delivery formulation are each a formulation for transdermal delivery, including, the transdermal delivery of an active ingredient for the treatment of a syndrome and or a disease in an individual.

For purposes herein, the terms lecithin and lecithin organogel are used interchangeably and both refer to, include and cover a lecithin organogel that comprises any group of yellow-brownish fatty substances occurring in animal and plant tissues which are amphiphilic and include a mixture of one or more of glycerophospholipids including phosphatidylcholine, phosphatidylethanolamine, phosphatidylinositol, phosphatidylserine, and phosphatidic acid.

Additionally, the use of particular formulations can disrupt the balance of electrolytes and cations, including those such as the Na/K ratio. For example, the administration of formulations containing calcium carbonate can reduce the amount of sodium or other ions which can decrease the potential for reaching a hyponatremic state. Also, the use of calcium carbonate can also increase the serum levels of calcium which can reduce the amount of calcium leeched from the body by high sodium concentrations.

The formulations and methods of use provided herein take these complexities of electrolyte balance into account. One approach utilized herein in making formulations that avoid electrolyte imbalance and cation overload is to use non-metal buffers or buffers without counterions. Suitable buffering agents for these embodiments include Lysine (free base), TRIS, and IEPA.

For transdermal topical administration in particular for agents other than buffer, a suitable formulation typically involves a penetrant that enhances penetration of the skin and is, in some embodiments, composed of chemical permeation enhancers (CPEs). In some cases, it can also include peptides designed to penetrate cells i.e. cell penetrating peptides (CPPs) also known as skin penetrating peptides (SPPs). The formulation may be applied for example in the form of topical lotions, creams, and the like, as described herein.

If the active agent is a buffer, the choice of buffer system is based on the criteria of capability of buffering at a suitable pH typically between 7 and 10.5, as well as biocompatibility of the buffer system itself and the compatibility of the buffer system with the remaining components of the formulation. Conversely, the formulation is chosen to be compatible with the buffer selected; amounts of penetrants are generally less than those advantageous for therapeutic agents in general.

The present disclosure herein demonstrates transdermal drug delivery, but avoids some of the negative effects on color, smell, grittiness and stability driven by the use of lecithin organogel, and further optimizes transdermal penetration

### Transdermal Delivery Formulation Components

Phosphatides—Soy lecithin contains about 57.5% w/w phosphatides. The primary phosphatides found in Soy Lecithin are inositol phosphatides (20.5% w/w of Soy lecithin), phosphatidylcholine (20%), and phosphatidylethanolamine (11% w/w of Soy lecithin). In some embodiments, phosphatidylcholine is used for the full amount (57.5% w/w of Soy lecithin) as it is known to aide in skin penetration. Other phosphatides include phosphatidic acid, phosphatidylserine and phosphatidylinositol.

In an embodiment, a transdermal delivery formulation contains a phosphatide in a concentration of at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Sterols—Soy lecithin contains about 2.5% w/w sterols. In some embodiments, benzyl alcohol is used in substitution of the sterol in a transdermal delivery formulation to act as a penetration enhancer. In another embodiment, a sterol is cholesterol, ergosterol, hopanoids, hydroxysteroid, phytosterol and/or other steroids.

In an embodiment, a transdermal delivery formulation contains a sterol or benzyl alcohol in a concentration of at least 1%, at least 2%, at least 3%, at least 4%, at least 5%, at least 6%, at least 7%, at least 8%, at least 9%, at least 10%, at least 15%, at least 20%, at least 25%, at least 30% or more w/w of the transdermal delivery formulation.

Carbohydrates—Soy lecithin contains about 5% w/w free carbohydrates. In some embodiments, glucose is used in substitution of a free carbohydrate to maintain the ratio of sugars in the transdermal delivery formulation disclosed herein. In another embodiment, a carbohydrate is a monosaccharide, a disaccharide, a polyol, a malto-oligosaccharide, an oligosaccharide, a starch, a polysaccharide. In a further embodiment, a carbohydrate is glucose, galactose, fructose, xylose, sucrose, lactose, maltose, trehalose, sorbitol, mannitol, maltodextrins, raffinose, stachyose, fructo-oligosaccharide, amylose, amylopectin, modified starches, glycogen, cellulose, hemicellulose, pectin and/or hydrocolloid.

In an embodiment, a transdermal delivery formulation contains a carbohydrate in a concentration of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Moisture—In some embodiments, the transdermal delivery formulation maintains the about 1% w/w of water contained in Soy lecithin.

In an embodiment, a transdermal delivery formulation contains water in a concentration of at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Fatty acids—Soy lecithin contains about 34% w/w fatty acids, including 18-19% w/w linoleic acid, 1-2% w/w alpha-linoleic acid, 8-9% w/w oleic acid, about 5% w/w Palmitic acid, and 1-2% w/w stearic acids. In some embodiments, the fatty acids are similar to the fatty acids contained in soy lecithin. In an embodiment, alpha-linoleic is removed from the transdermal delivery formulation as it is known to oxidize and can become rancid. In some embodiments, the amount of stearic acid has been increased (i.e., enhancing with stability of the formulation) or linoleic acid (i.e., enhances skin penetration). In some embodiments, a seed oil such as purified safflower oil is used in a transdermal delivery formulation due to its similarity to the fatty acids found in Soy lecithin, its relative availability and its low cost. In some embodiments, the fatty acid content of a transdermal formulation can be adjusted with a different seed oil through the addition of smaller amounts of the fatty acids disclosed herein.

In an embodiment, a transdermal delivery formulation contains a carbohydrate in a concentration of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In a further embodiment, a fatty acid is a saturated or an unsaturated fatty acid. In another embodiment, an unsaturated fatty acid is myristoleic acid, palmitoleic acid, sapienic acid, oleic acid, elaidic acid, vaccenic acid, linoleic acid, linoelaidic acid, α-Linolenic acid, arachidonic acid, eicosapentaenoic acid, erucic acid and/or docosahexaenoic acid. In an embodiment, a saturated fatty acid is caprylic acid, capric acid, lauric acid, myristic acid, palmitic acid, stearic acid, arachidic acid, behenic acid, lignoceric acid and/or cerotic acid. In another embodiment, the fatty acid is a dietary fat and include duct fat, lard, tallow, butter, coconut oil, cocoa butter, palm kernel oil, palm oil, cottonseed oil, wheat germ oil, soybean oil, olive oil, corn oil, sunflower oil, safflower oil, help oil and/or canola/rapeseed oil.

In some embodiments, carotenoids are excluded from the formulations disclosed herein.

Herein we describe formulations demonstrating the replacement of lecithin organogel (i.e., Lecithin and Isopropyl Palmitate).

In an embodiment, a transdermal delivery formulation comprises the components of Table 1:

TABLE 1

| Ingredient | Weight % |
|---|---|
| Phosphatidylcholine | 28.75% |
| Glucose | 2.50% |
| Benzyl Alcohol | 1.25% |
| Deionized water | 0.50% |
| Linoleic Acid | 9.75% |
| Oleic Acid | 4.38% |
| Stearic Acid | 2.88% |
| Isopropyl Palmitate | 50.00% |

In some another embodiment, a transdermal delivery formulation comprises the components of Table 2:

TABLE 2

| Ingredient | Weight % |
|---|---|
| Phosphatidylcholine | 28.75% |
| Glucose | 2.50% |
| Benzyl Alcohol | 1.25% |
| Deionized water | 0.50% |
| Safflower Oil | 11.06% |
| Oleic Acid | 3.65% |
| Stearic Acid | 2.34% |
| Isopropyl Palmitate | 50.00% |

In an aspect, the concentration of Phosphatidylcholine in a transdermal delivery formulation is at least 10%, at least 15%, at least 20%, at least 25%, at least 28.75%, at least 30%, at least 35%, at least 40% or more. In an aspect, the concentration of Phosphatidylcholine in a transdermal delivery formulation is not more than 10%, not more than 15%, not more than 20%, not more than 25%, not more than 28.75%, not more than 30%, not more than 35%, not more than 40% or more. In an aspect, the concentration of Phosphatidylcholine in a transdermal delivery formulation is about 10%, about 15%, about 20%, about 25%, at least 28.75%, about 30%, about 35%, about 40% or more. In an aspect, the concentration of Phosphatidylcholine in a transdermal delivery formulation is from 10% to 40%, is from 15% to 35%, is from 20% to 30%, is from 25% to 30%, is from 28% to 29%.

In another aspect, the concentration of Glucose in a transdermal delivery formulation is at least 1%, at least 2%, at least 2.5%, at least 3%, at least 4%, at least 5%, at least 6%, at least 7%, at least 8%, at least 9% or more. In another aspect, the concentration of Glucose in a transdermal delivery formulation is about 1%, about 2%, about 2.5%, about 3%, about 4%, about 5%, about 6%, about 7%, about 8%, about 9% or more. In another aspect, the concentration of Glucose in a transdermal delivery formulation is no more than 1%, no more than 2%, no more than 2.5%, no more than 3%, no more than 4%, no more than 5%, no more than 6%, no more than 7%, no more than 8%, no more than 9% or more. In another aspect, the concentration of Glucose in a transdermal delivery formulation is from 1% to 10%, is from 2% to 9%, is from 2.5% to 5%, is from 2% to 3%, is from 3% to 8%, if from 4% to 7%, if from 5% to 6%, is from 2% to 4%, if from 1.5% to 3.55. In an embodiment, a transdermal delivery formulation contains no glucose. In another embodiment, a transdermal delivery formulation does not contain a carbohydrate.

In a further embodiment, the concentration of Benzyl Alcohol in a transdermal formulation is at least 0.25%, at least 0.5%, at least 0.75%, at least 1%, at least 2%, at least 2.5%, at least 3%, at least 4%, at least 5% or more. In an embodiment, the concentration of Benzyl Alcohol in a transdermal formulation is about 0.25%, about 0.5%, about 0.75%, about 1%, about 2%, about 2.5%, about 3%, about 4%, about 5% or more. In another embodiment, the concentration of Benzyl Alcohol in a transdermal formulation is at from 0.25% to 5%; from 0.5% to 4%, from 0.75% to 3%, from 1% to 2.5% or from 0.5% to 2%. In a further embodiment, the concentration of Benzyl Alcohol in a transdermal formulation is no more than 0.25%, no more than 0.5%, no more than 0.75%, no more than 1%, no more than 2%, no more than 2.5%, no more than 3%, no more than 4%, no more than 5%.

In an embodiment, the concentration of Deionized Water in a transdermal formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5% or more. In an embodiment, the concentration of Deionized Water in a transdermal formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5% or more. In an embodiment, the concentration of Deionized Water in a transdermal formulation is from 0.1% to 5%, from 0.2% to 4%, from 0.3% to 3%, 0.4%-2%, 0.5% to 1%, from 0.6% t 0.9%, from 0.7% to 0.8%, from 0.4% to 1.5%, from 0.3% to 0.7% or from 0.4% to 0.6%. In an embodiment, the concentration of Deionized Water in a transdermal formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5% or more.

In an aspect, the concentration of Safflower oil in a transdermal delivery formulation is at least 1%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of Safflower oil in a transdermal delivery formulation is about 1%, about 5%, about 7.5%, about 10%, about 11%, about 11.06%, about 12%, about 13%, about 14%, about 15%, about 16%, about 17%, about 18%, about 19%, about 20% or more. In an aspect, the concentration of Safflower oil in a transdermal delivery formulation is from 1% to 20%, from 5% to 19%, from 7.5% to 18%, from 10% to 17%, from 11% to 16%, from 11.06%, 12% from 11% to 12%, from 12% to 14%, from 13% to 14%, from 10% to 12%, from 10.5% to 12.5% or from 11% to 11.25%. In an aspect, the concentration of Safflower oil in a transdermal delivery formulation is no more than 1%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, no more than 20%, no more than or more.

In a further aspect, the concentration of Oleic Acid in a transdermal delivery formulation is at least 1%, at least 2%, at least 3%, at least 3.65%, at least 4%, at least 5%, at least 6%, at least 7%, at least 8%, at least 9%, at least 10% or more. In a further aspect, the concentration of Oleic Acid in a transdermal delivery formulation is about 1%, about 2%, about 3%, about 3.65%, about 4%, about 5%, about 6%, about 7%, about 8%, about 9%, about 10% or more. In a further aspect, the concentration of Oleic Acid in a transdermal delivery formulation is no more than 1%, no more than 2%, no more than 3%, no more than 3.65%, no more than 4%, no more than 5%, no more than 6%, no more than 7%, no more than 8%, no more than 9%, no more than 10% or more. In another aspect, the concentration of Stearic Acid in a transdermal formulation is from 1% to 10%, from 2% to 9%, from 2% to 3%, from 3% to 4%, from 3% to 8%, from 4% to 7%, from 5% to 6%, from 2 to 2.5% or from 2.5% to 4%.

In another aspect, the concentration of Stearic Acid in a transdermal formulation is at least 1%, at least 2%, at least 2.34%, at least 3%, at least 4%, at least 5%, at least 6%, at least 7%, at least 8%, at least 9%, at least 10% or more. In another aspect, the concentration of Stearic Acid in a transdermal formulation is no more than 1%, no more than 2%, no more than 2.34%, no more than 3%, no more than 4%, no more than 5%, no more than 6%, no more than 7%, no more than 8%, no more than 9%, no more than 10% or more. In another aspect, the concentration of Stearic Acid in a transdermal formulation is about 1%, about 2%, about 2.34%, about 3%, about 4%, about 5%, about 6%, about 7%, about 8%, about 9%, about 10% or more. In another aspect, the concentration of Stearic Acid in a transdermal formulation is from 1% to 10%, from 2% to 9%, from 2% to 3%, from 2.34% to 2.5%, from 3% to 8%, from 4% to 7%, from 5% to 6% or from 1.5% to 2.5%.

In an aspect, the concentration of Isopropyl Palmitate in a transdermal formulation is at least 10%, at least 20%, at least 25%, at least 30%, at least 40%, at least 45%, at least 50%, at least 55%, at least 60%, at least 65%, at least 70%, at least 75% or more. In an aspect, the concentration of Isopropyl Palmitate in a transdermal formulation is about 10%, about 20%, about 25%, about 30%, about 40%, about 45%, about 50%, about 55%, about 60%, about 65%, about 70%, about 75% or more. In an aspect, the concentration of Isopropyl Palmitate in a transdermal formulation is no more than 10%, no more than 20%, no more than 25%, no more than 30%, no more than 40%, no more than 45%, no more than 50%, no more than 55%, no more than 60%, no more than 65%, no more than 70%, no more than 75% or more. In an aspect, the concentration of Isopropyl Palmitate in a transdermal formulation is from 10% to 75%, from 20% to 70%, from 25% to 65%, from 30% to 60%, from 40% to 55%, from 45% to 50%, from 40% to 60%, from 45% to 55% or from 47% to 53%.

In another aspect, certain embodiments of a transdermal delivery formulation use buffers which do not have counter ions and thus have reduced or eliminated the risk of hypernatremia. Tris-base buffers have other potentially beneficial characteristics including a demonstrated antitumor effect in vivo. Accordingly, certain embodiments of the formulation incorporate a Tris-base in an amount of up to about 60.0% w/w; up to about 50.0% w/w; up to about 45.0% w/w; up to about 40.0% w/w; up to about 35.0% w/w; up to about 30.0% w/w; up to about 25.0% w/w; up to about 20.0% w/w; up to about 17.0% w/w; up to about 15.0% w/w; up to about 10.0% w/w; or up to about 5.0% w/w.

Certain components or ingredients of transdermal delivery formulation provided herein may be supplemented with components described in greater detail in the inventor's related applications mentioned above, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

Iron

A transdermal delivery formulation containing iron may be formulated at acidic pH to minimize the spontaneous oxidation Fe(II) into Fe(III).

Suitable nonlimiting exemplary iron chelators include deferoxamine, ethylenediaminetetraacetic acid (EDTA), 1,2-diethyl-3-hydroxypyridin-4-one (CP94), Desferol, Deferiprone and Deferasirox, succimer, trientine, Desferrithiocin, Clioquinol, O-trensox, Tachpyr, Dexrazoxane, Triapine, Pyridoxal isonicotinoyl hydrazone, Di-2-pyridylketone thiosemicarbazone series, Flavan-3-ol, Curcumin, Apocynin, Kolaviron, Floranol, Baicalein, Baicalin, ligustrazine, Quercetin, Epigallocatechin gallate, Theaflavin, Phytic acid, and Genistein.

Suitable nonlimiting exemplary antioxidants include glutathione, vitamin C, vitamin E, superoxide dismutase, catalase, pNaKtide, Butylated hydroxytoluene, Butylated hydroxyanisole, tert-Butylhydroquinone, HP beta CD, resveratrol, retinol, coenzyme q10, niacinamide, polyphenols, flavenoids, beta-carotene, lutein, and lycopene.

A transdermal delivery formulation comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

In some embodiments, a transdermal delivery formulation comprises phosphatidylcholine in amount less than 12% w/w or 18% w/w of the formulation. In some embodiments, the transdermal delivery formulation comprises a phospholipid in amount less than 12% w/w or 18% w/w of the formulation. In some embodiments, the transdermal delivery formulation comprises a mixture of tridecane and undecane in amount less than 2% w/w, 5 w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the transdermal delivery formulation comprises cetyl alcohol in amount less than 2% w/w, 5% w/w, or 8% w/w of the formulation. In some embodiments, the transdermal delivery formulation comprises benzyl alcohol in an amount less than about 2% w/w, 5% w/w, or 8% w/w. In some embodiments, the transdermal delivery formulation comprises stearic acid in an amount less than 2% w/w, 5% w/w, or 8% w/w of the formulation.

In any of the anesthetic compositions of a transdermal delivery formulation, it may be desirable to administer the epinephrine in tandem with a transdermal anesthetic. Alternatively, treatment of the epinephrine with a chelator, such as the iron chelator Desferal® may stabilize the epinephrine sufficiently to include it in the transdermal delivery formulation.

A suitable dose of iron or an iron containing transdermal delivery formulation administered topically as a transdermal delivery formulation for a subject (e.g. a human patient) is at least about 500 mg, at least about 750 mg, at least about 1000 mg, at least about 1.5 g, at least about 2.0 g, at least about 2.5 g, at least about 3.0 g, at least about 3.5 g, at least about 4.0 g, at least about 4.5 g, at least about 5.0 g, at least about 6.0 g, at least about 7.0 g, at least about 8.0 g, at least about 9.0 g, at least about 10.0 g, at least about 11 g, at least about 12 g, at least about 13 g, at least about 14 g, at least about 15 g, at least about 20 g, at least about 25 g, at least about 30 g, at least about 35 g, at least about 40 g, at least about 45 g, at least about 50 g, or more. This dose is typically administered daily, twice a day, or three times a day, but it may also be administered four times a day, five times a day, or more than five times a day.

Alternatively, a suitable daily dose of iron or an iron containing transdermal delivery formulation administered topically as a transdermal delivery formulation for a subject is at least about 10 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, up to at least about 500 mg/kg or more.

In another aspect, a suitable daily dose of iron or an iron containing transdermal delivery formulation administered topically as a transdermal delivery formulation for a subject is about 10 mg/kg to about 1.0 g/kg, and more typically the daily dose is about 10 mg/kg to about 500 mg/kg, about 25 mg/kg to about 500 mg/kg, about 50 mg/kg to about 300 mg/kg, about 75 mg/kg to about 300 mg/kg, about 75 mg/kg to about 250 mg/kg, about 100 mg/kg to about 300 mg/kg, about 75 mg/kg to about 200 mg/kg, about 100 mg/kg to about 200 mg/kg, or alternative ranges.

In one aspect, disclosed herein is a transdermal delivery formulation for transdermal delivery ketone components through the skin of a subject, comprising: a ketone component in an amount between about 10-60% w/w; a transdermal delivery formulation in an amount less than about 60% w/w, and water in an amount less than about 50% w/w.

In another aspect disclosed herein is a method of inducing ketosis to treat a disorder and/or treating a disorder with ketone supplementation in a subject, wherein the method comprises administering an effective amount of a transdermal delivery formulation for transdermal delivery of one or more ketone components through the skin of a subject, comprising: a ketone component in an amount between about 10-60% w/w; a transdermal delivery formulation in an amount less than about 60% w/w, and water in an amount less than about 50% w/w. Particularly suitable formulation for transdermal delivery ketone components are described in the U.S. Application No. 62/742,172, filed Oct. 5, 2018, which is incorporated by reference herein.

A transdermal delivery formulation of the disclosure may be prepared in a number of ways. Typically, the components of a transdermal delivery formulation are simply mixed together in the required amounts. However, it is also desirable in some instances to, for example, to carry out partial dissolution of a ketone component and then add a separate preparation containing the components aiding the delivery of the ketones in the form of a carrier. The concentrations of these components in the carrier, then, will be somewhat higher than the concentrations required in the final transdermal delivery formulation. Thus, a ketone component may first be partially dissolved in water and then added to a carrier comprising an alcohol, transdermal delivery formulation and optionally a combination of a nonionic surfactant and polar gelling agent, or of ionic detergent. Alternatively, some subset of these components can first be mixed and then “topped off” with the remaining components either simultaneously or sequentially. The precise manner of preparing a transdermal delivery formulation will depend on the choice of ketone components and the percentages of the remaining components that are desirable with respect to that ketone component. In some embodiments, the water is less than about 85% w/w, 50% w/w, or 30% w/w of the transdermal delivery formulation.

In some embodiments, the one or more ketone components are formulated with Aveeno® moisturizers, cream, oils, lotions; Jergens® moisturizers, cream, oils, lotions; Honest Company® moisturizers, cream, oils, lotions; Dermologica® moisturizers, cream, oils, lotions; or St. Ives™ moisturizers, cream, oils, lotions. In some embodiments, the commercial lotions, moisturizers, etc. are formulated with the ketone component in an amount between about 10-60% w/w or at least 10% w/w, at least 20% w/w, at least 30% w/w, at least 40% w/w, at least 50% w/w, at least 60% w/w, at least 75% w/w or more.

The transdermal delivery formulation is a multi-component mixture, whereby the particular concentrations of the penetration enhancers are informed in part by the particle size of the ketone component. The formulation enables the ketone component to become bio-available to the target site within minutes of topical administration. In some embodiments, the transdermal delivery formulation comprises an alcohol in an amount less than 5% w/w of the formulation.

Subjects of the disclosure herein, in addition to humans, include veterinary subjects, wherein formulations suitable for these subjects are also appropriate. Such subjects include livestock and pets as well as sports animals such as horses and greyhounds.

One aspect of the invention is a method to inhibit cancer growth and metastasis, including diminution of cancer mass by non-systemic parenteral, including topical administration of ketone components as disclosed herein, including solid tumors and melanomas.

A transdermal delivery formulation comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

For topical administration, and in particular transdermal administration, a transdermal delivery formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290 ('290), WO2014/209910 ('910), and WO2017/127834. In addition to transdermal delivery formulations with penetrants, transdermal delivery can be affected by mechanically disrupting the surface of the skin to encourage penetration, or simply by supplying the formulation applied to the skin under an occlusive patch.

Alternatively, the transdermal delivery formulation comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Suitable gelling components also include isopropyl palmitate, ethyl laurate, ethyl myristate and isopropyl myristate. In some embodiments, a transdermal delivery formulation comprises a gelling agent in an amount less than 5% w/w of a transdermal delivery formulation. Certain hydrocarbons, such as cyclopentane, cyclooctane, trans-decalin, trans-pinane, n-pentane, n-hexane, n-hexadecane may also be used. In some embodiments, the transdermal delivery formulation comprises a mixture of xanthan gum, sclerotium gum, pullulan, or a combination thereof in an amount less than 2% w/w, 5% w/w, or 10% w/w of the formulation. In some embodiments, a transdermal delivery formulation comprises Siligel™ in an amount between about 1-5% w/w or 5-15% w/w, or an equivalent mixture of xanthan gum, sclerotium gum, and pullulan. In some embodiments, a transdermal delivery formulation comprises a mixture of caprylic triglycerides and capric triglycerides in amount less than 2% w/w, 8% w/w, or 10% w/w of the formulation. In some embodiments, a transdermal delivery formulation comprises Myritol® 312 in an amount between about 0.5-10% w/w, or an equivalent mixture of caprylic triglycerides and capric triglycerides.

In some embodiments, a transdermal delivery formulation is in an amount between about 10-90% w/w or 10-50% w/w of the formulation or at least 10% w/w, at least 20% w/w, at least 30% w/w, at least 40% w/w, at least 50% w/w, at least 60% w/w, at least 70% w/w, at least 80% w/w, at least 90% w/w or at least 95% w/w. In some embodiments, a transdermal delivery formulation comprises phosphatidyl choline in amount less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w or less than 18% w/w of the formulation. In some embodiments, a transdermal delivery formulation comprises a phospholipid in amount less than 20% w/w, less than 30% w/w, less than 40 w/w, less than or 50% w/w of the formulation. In some embodiments, a transdermal delivery formulation comprises a mixture of tridecane and undecane in amount less than 2% w/w, 3% w/w, 4% w/w, 5% w/w, 6% w/w, 7% w/w, or 8% w/w of the formulation. In some embodiments, the formulation comprises Cetiol Ultimate® in an amount less than about 2% w/w, 3% w/w, 4% w/w, 5% w/w, 6% w/w, 7% w/w, 8% w/w, 9% w/w, or 10% w/w, or an equivalent mixture of tridecane and undecane. In some embodiments, a transdermal delivery formulation comprises cetyl alcohol in amount less than 2% w/w, 3% w/w, 4% w/w, 5% w/w, 6% w/w, 7% w/w, 8% w/w, 9% w/w, or 10% w/w of the formulation. In some embodiments, the formulation comprises benzyl alcohol in an amount less than about 2% w/w, 3% w/w, 4% w/w, 5% w/w, 6% w/w, 7% w/w, 8% w/w, 9% w/w, or 10% w/w. In some embodiments, a transdermal delivery formulation comprises stearic acid in an amount less than 2% w/w, 3% w/w, 4% w/w, 5% w/w, 6% w/w, 7% w/w, 8% w/w, 9% w/w, or 10% w/w of the formulation. In some embodiments, the transdermal delivery formulation comprises phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 30% w/w or in amount less than 12% w/w of the formulation.

An additional component in a transdermal delivery formulation of the disclosure is an alcohol. Benzyl alcohol and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 53% w/w, 4% w/w, 5% w/w, 6% w/w, 7% w/w, 8% w/w, 9% w/w, or 10% w/w, and other intermediate weight percentages are included. Due to the aromatic group present in a transdermal delivery formulation such as benzyl alcohol, the molecule has a polar end (the alcohol end) and a non-polar end (the benzene end). This enables the agent to dissolve a wider variety of transdermal delivery formulation components.

In some embodiments, as noted above, the performance of a transdermal delivery formulation is further improved by including a nonionic detergent and polar gelling agent or including a powdered surfactant. In both aqueous and anhydrous forms of the composition, detergents, typically nonionic detergents are added. In general, the nonionic detergent should be present in an amount between about 1% w/w to 30% w/w of a transdermal delivery formulation. Typically, in the compositions wherein a transdermal delivery formulation is topped off with a polar or aqueous solution containing detergent, the amount of detergent is relatively low—e.g., 2-25% w/w, or 5-15% w/w or 7-12% w/w of a transdermal delivery formulation. However, in compositions that are essentially anhydrous and are topped-off by powdered detergent, relatively higher percentages are usually used—e.g., 20-60% w/w.

In some embodiments, a transdermal delivery formulation further comprises a detergent portion in an amount between about 1 to 70% w/w or 1-60% w/w of a transdermal delivery formulation. In some embodiments, the nonionic detergent provides suitable handling properties whereby the formulations are gel-like or creams at room temperature. To exert this effect, the detergent, typically a poloxamer, is present in an amount between about 2-12% w/w of a transdermal delivery formulation, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a transdermal delivery formulation also contains surfactant, typically, nonionic surfactant at 2-25% w/w of a transdermal delivery formulation along with a polar solvent wherein the polar solvent is present in an amount at least in molar excess of the nonionic surfactant. In these embodiments, typically, the composition comprises the above-referenced amounts of a transdermal delivery formulation and benzyl alcohol along with a ketone component with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount between about 1-30% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about I % w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that may also be included in a transdermal delivery formulation are fatty acids, terpenes, lipids, and cationic, and anionic detergents. In some embodiments, a transdermal delivery formulation further comprises tranexamic acid in an amount less than 2% w/w, 5% w/w, or 10% w/w of the formulation. In some embodiments, a transdermal delivery formulation further comprises a polar solvent in an amount less than 2% w/w, 5% w/w, 10% w/w, or 20% w/w of the transdermal delivery formulation. In some embodiments, a transdermal delivery formulation further comprises a humectant, an emulsifier, an emollient, or a combination thereof. In some embodiments, a transdermal delivery formulation further comprises almond oil in an amount less than about 5% w/w. In some embodiments, a formulation further comprises a mixture of thermoplastic polyurethane and polycarbonate in an amount less than about 5% w/w. In some embodiments, a transdermal delivery formulation further comprises phosphatidylethanolamine in an amount less than about 5% w/w. In some embodiments, a transdermal delivery formulation further comprises an inositol phosphatide in an amount less than about 5% w/w.

Other solvents and related compounds that may be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid⋅ include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that may be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants may be included, such as vitamin C, vitamin E, proanthocyanidin and a-lipoic acid typically in concentrations of 0.1%-2.5% w/w.

In some applications, it is desirable to adjust the pH of a transdermal delivery formulation to assist in permeation or to adjust the nature of the ketone component and/or of the target compounds in the subject. In some instances, the pH is adjusted to a level of pH 9-11 or 10-11 which can be done by providing appropriate buffers or simply adjusting the pH with base.

In some applications, in particular when a transdermal delivery formulation includes an anesthetic, epinephrine or an alternate vasoconstrictor, such as phenylephrine or epinephrine sulfate may be included in the formulation if a stabilizing agent is present. Otherwise, the epinephrine should be administered in tandem since epinephrine is not stable at alkali pH.

Another active agent is Withaferin A. Withaferin A inhibits tumor metastasis and manifests other anti-cancer activities, e.g., inhibition of the neovascularzation associated with carcinoma, as well as cell proliferation. Withaferin A is also a leptin sensitizer with strong anti-diabetic properties that could induce healthy weight loss and beneficial effects on glucose metabolism.

Other agents include anti-metastatic agents including inhibitors of the src homology region 2-containing protein tyrosinase phosphatase (Shp2). A multiplicity of inhibitors of this activity is known, including Fumosorine, PHPS (NSC-87877) and NSC-117199, phenylhydrazonopyrazolone sulfonate (PHPS1), DCA, cryptotanshinone, 11-B08 and #220-324, metalloproteinases-2 and -9 (MMP-2 and MMP-9) and certain cathepsins, in particular B, D and L.

Other agents include inhibitors of E-cadherin and of epidermal growth factor receptor (EGFR). Known inhibitors include erlotinib, an anti-integrin drug (Cilengitide), Cariporide, Eniporide and Amiloride.

A transdermal delivery formulation may include other components that act as excipients or serve purposes other than active anti-tumor effects. For example, preservatives like antioxidants e.g., ascorbic acid or α-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft® (which is a mixture of thermoplastic polyurethane and polycarbonate). Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a transdermal delivery formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In an embodiment, a transdermal delivery formulation comprises the components of Table 5:

| Ingredient | Weight Percent (%) |
|---|---|
| Deionized Water | 38.40% |
| Dextrose Anhydrous | 0.65% |
| Phospholipon 90G | 7.47% |
| Isopropyl Palmitate | 13.00% |
| Benzyl Alcohol | 1.36% |
| Stearic Acid | 0.61% |
| Safflower Oil | 2.87% |
| Oleic Acid | 0.95% |
| Limonene | 1.00% |
| Crystalline Cannabidiol | 2.50% |
| Durosoft PK-SG | 1.04% |
| 30% Pluronic Gel (Dyve) | 30.15% |

A wide variety of therapeutic agents may be used in a transdermal delivery formulation, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply effect transport across the skin into a localized subdermal location, such as treatment of nail fungus or modulation of hair growth or may effect systemic delivery such as is desirable in some instances where vaccines are used.

In addition to the compositions and formulations of the invention per se, the methods may employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the ketone component to pass through the epidermis to the dermis at least, and, if systemic administration is achieved, through the dermis itself.

For administration of anesthetics as the therapeutic agent, the local anesthetic may be one or more of the following: benzocaine, lidocaine, tetracaine, bupivacaine, cocaine, etidocaine, mepivacaine, pramoxine, prilocaine, procaine, chloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof. Combinations of anesthetic agents may also be used. The anesthetic agent(s) are included in the composition in effective amount(s). Depending on the anesthetic(s) the amounts of anesthetic or combination is typically in the range of 1% w/w to 50% w/w. The compositions of the invention provide rapid, penetrating relief that is long lasting. The pain to be treated can be either traumatic pain and/or inflammatory pain.

In one embodiment, the anesthetic is administered to relieve the pain associated with invasive fat deposit removal. Specific removal of fat deposits has been attractive for both health and cosmetic reasons. Among the methods employed are liposuction and injection of a cytolytic agent for fat such as deoxycholic acid (DCA). For example, a series of patents issued or licensed to Kythera Biopharmaceuticals is directed to methods and compositions for non-surgical removal of localized fat that involves injecting compositions containing DCA or a salt thereof. Representative issued patents are directed to formulation (U.S. Pat. No. 8,367,649); method-of-use (U.S. Pat. Nos. 8,846,066; 7,622,130; 7,754,230; 8,298,556); and synthetic DCA (7,902,387).

In this aspect of the invention, conventional invasive fat removal techniques are employed along with administering a pain-relieving effective agent—typically lidocaine or related anesthetics via transdermal administration. In some embodiments, the pain-relieving transdermal formulation is applied to the area experiencing pain immediately before, during or immediately after the invasive fat-removal procedure.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

The compositions containing anesthetics are useful for temporary relief of pain and itching associated with minor burns, cuts, scrapes, skin irritations, inflammation and rashes due to soaps, detergents or cosmetics, or, as noted above, pain associated with removal of fat deposits.

In another embodiment, nutrients are supplied via transdermal administration. There are many occasions in which the formulations of the invention are useful. For athletes, a transdermal delivery formulation can deliver to tired muscles sufficient amounts of a neutralizing agent for lactic acid, such as ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation especially in those areas of their musculature that are involved in performing workouts and, therefore, need to consume large numbers of calories. These nutrients can be supplied directly rather than requiring oral ingestion which is counterproductive and relatively slow.

Emergency medical treatment of individuals requiring, for example, blood balancing agents including electrolytes and readily-metabolized nutrients, such as glucose, that would otherwise be administered intravenously can instead be non-invasively treated by massaging the formulation through the skin and thus permitting systemic delivery so that levels in the bloodstream are altered.

In addition to these applications, it has been noted that the administration of nutrients according to the invention also assuages feelings of hunger. Therefore, a transdermal delivery formulation of the invention and methods of the invention are useful in promoting weight loss as the caloric intake required to assuage feelings of hunger is lower than that ordinarily experienced by consuming food conventionally. Thus, in addition to individuals requiring extra calories or metabolic balancers because of exertion and in addition to those unable to feed themselves orally, suitable subjects for the methods of the invention include individuals seeking to control their caloric intake in order to adjust their weight. In view of the generally acknowledged obesity epidemic in the United States in particular, this is an important group of subjects benefitting from the methods of the invention.

It is clear that the nature of the desired. ingredients will vary depending on the object of the administration. Simple nutrients such as amino acids, glucose, fructose, simple fats, various vitamins, cofactors and antioxidants as well as somewhat more complex foodstuffs can be administered as well as neutralizing agents, depending on the need.

In some embodiments, the components for athletic performance include beta-alanine, L-carnitine, adenosine triphosphate, dextrose, creatine monohydrate, beta hydroxy-betamethylbutyrate (HMB), branched chain amino acids (leucine, isoleucine, valine), glutathione, sodium phosphate, and caffeine. Components for medical nutrition include amino acids, dextrose, lipids, Na⁺, K⁺, Ca²⁺, Mg²⁺, acetate, Cl⁻, P, multivitamin, and trace elements. While components for weight loss include conjugated linoleic acids, ephedra, caffeine, and salicin.

Certain embodiments of a transdermal delivery formulation provided herein may be supplemented with formulation components described in greater detail in the inventor's related applications, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

In some particular embodiments it is desirable to adjust the pH of a transdermal delivery formulation and the pH is adjusted to a level of pH 9-11 or 10-11, which can be done by providing appropriate buffers or simply adjusting the pH with base. In other embodiments, it is desirable to adjust the pH of a transdermal delivery formulation to a level of pH 4-6, which can be done by providing appropriate buffers or simply adjusting the pH with an acid.

In some applications a formulation for transdermal delivery may, for example, comprise: Aveeno®, for example in an amount between about 10-95% w/w; between about 20-85% w/w, between about 20-75% w/w, between about 20-50% w/w.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

The formulation described in this specification may also comprise more than one therapeutic compound as desired for the particular indication being treated, preferably those with complementary activities that do not adversely affect the other proteins. A transdermal delivery formulation to be used for in vivo administration can be sterile. This can be accomplished, for instance, without limitation, by filtration through sterile filtration membranes, prior to, or following, preparation of a transdermal delivery formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

Methods

Methods for treating, preventing or ameliorating a disease, disorder, a condition, or a symptom thereof or a condition related thereto are provided herein using a transdermal delivery formulation for transdermal delivery described herein below. The methods provided herein may comprise or consist of topically administering one or more of a transdermal delivery formulation described herein to skin of a subject in need thereof. Preferred, but non-limiting embodiments are directed to methods for treating, preventing, inhibiting or ameliorating a disease, disorder, a condition, or a symptom described below.

### Caners and Tumors

Many embodiments provided herein are directed to various methods of treating cancer and/or tumors. An exemplary embodiment of a method of treating cancer in a patient according to the invention comprises administering topically and/or transdermally an effective amount of a transdermal delivery formulation comprising one or more buffering agent to a patient in need thereof, wherein said administration is effective to inhibit or prevent the growth of a tumor or tumor cells.

Another embodiment is directed to a method of preventing metastasis of tumors comprising administering topically and/or transdermally an effective amount of a transdermal delivery formulation comprising one or more buffering agent to a patient in need thereof, where the administration is effective to inhibit or prevents the metastasis of tumors or cancer cells.

Another embodiment is directed to a method of preventing the intravasation of tumor cells comprising administering topically and/or transdermally an effective amount of a transdermal delivery formulation comprising one or more buffering agent to a patient in need thereof, where the administration is effective to inhibit or prevent the intravasation of tumor cells.

Another embodiment is directed to a method of treatment of cancer, the method comprising i) selecting a therapeutic agent (e.g. a chemotherapeutic of immunotherapeutic agent) described herein and formulating the therapeutic agent in a transdermal delivery formulation comprising one or more buffering agent, and iii) administering the formulation topically and/or transdermally in an amount effective to inhibit or prevent the growth of a tumor or tumor cells.

Another embodiment is directed to a method of improving, extending the duration of remission, or maintaining remission of a cancer or tumor comprising administering topically and/or transdermally an effective amount of a transdermal delivery formulation comprising one or more buffering agent to a patient in need thereof, where administration is effective to improve, extend the duration of remission, or maintain remission of a cancer or tumor.

In other embodiments, a method of treating cancer in a patient comprises administering topically and/or transdermally an effective amount of a transdermal delivery formulation comprising one or more buffering agent to a patient in need thereof, where the administration is effective to alter the pH of a tissue or microenvironment proximal to a solid tumor or cancer cells in the patient, wherein the change in the pH of a tissue or microenvironment proximal to a solid tumor or cancer cells inhibits the growth of said solid tumor or cancer cells.

In other embodiments, a method of altering the pH of a tissue or microenvironment proximal to a solid tumor or cancer cells in a patient is provided. These embodiments generally comprise administering topically and/or transdermally an effective amount of a transdermal delivery formulation comprising one or more buffering agent to a patient in need thereof, wherein the administration is effective to alter the pH of a tissue or microenvironment proximal to a solid tumor or cancer cells in the patient.

In other embodiments, a method of inhibiting or preventing the metastasis of tumors in a patient is provided. These embodiments generally comprise administering topically and/or transdermally an effective amount of a transdermal delivery formulation comprising one or more buffering agent to a patient in need thereof, wherein the administration is effective to alter the pH of a tissue or microenvironment proximal to a solid tumor or cancer cells in the patient, and where the change in the pH of a tissue or microenvironment proximal to a solid tumor or cancer cells inhibits or prevents the metastasis of tumors or cancer cells.

In other embodiments, a method of inhibiting or preventing the intravasation of tumor cells in a patient is provided. These embodiments generally comprise administering topically and/or transdermally an effective amount of a transdermal delivery formulation comprising one or more buffering agent to a patient in need thereof, wherein the administration is effective to inhibit or prevent the intravasation of tumor cells.

Formulations provided herein are used in methods of treating many cancers, including but not limited to breast cancer, prostate cancer, pancreatic cancer, lung cancer, bladder cancer, skin cancer, colorectal cancer, kidney cancer, liver cancer, and thyroid cancer.

Formulations provided herein are also used in methods of treating a cancer or tumor, including but not limited to Adrenocortical Carcinoma, Basal Cell Carcinoma, Bladder Cancer, Bone Cancer, Brain Tumor, Breast Cancer, Cervical Cancer, Colon Cancer, Colorectal Cancer, Esophageal Cancer, Retinoblastoma, Gastric (Stomach) Cancer, Gastrointestinal Tumors, Glioma, Head and Neck Cancer, Hepatocellular (Liver) Cancer, Islet Cell Tumors (Endocrine Pancreas), Kidney (Renal Cell) Cancer, Laryngeal Cancer, Non-small Cell Lung Cancer, Small Cell Lung Cancer, Medulloblastoma, Melanoma, Pancreatic Cancer, Prostate Cancer, Renal Cancer, Rectal cancer, and Thyroid Cancer.

While preferred embodiments of the methods provided herein are typically directed to a particular cancer, solid tumor or grouping thereof, a more complete but still non-limiting listing of suitable cancers and tumors that may be tested for effectiveness according to embodiments provided herein includes the following: lymphoblastic leukemia (ALL), acute myeloid leukemia (AML), adrenocortical carcinoma, aids-related cancers, kaposi sarcoma (soft tissue sarcoma), aids-related lymphoma (lymphoma), primary cns lymphoma (lymphoma), anal cancer, astrocytomas, atypical teratoid/rhabdoid tumor, childhood, central nervous system (brain cancer), basal cell carcinoma, bile duct cancer, bladder cancer. childhood bladder cancer, bone cancer (includes ewing sarcoma and osteosarcoma and malignant fibrous histiocytoma), brain tumors, breast cancer, childhood breast cancer, bronchial tumors, burkitt lymphoma (non-hodgkin lymphoma, carcinoid tumor (gastrointestinal), childhood carcinoid tumors, cardiac (heart) tumors, central nervous system tumors. atypical teratoid/rhabdoid tumor, childhood (brain cancer), embryonal tumors, childhood (brain cancer), germ cell tumor (childhood brain cancer), primary cns lymphoma, cervical cancer, childhood cervical cancer, cholangiocarcinoma, chordoma (childhood), chronic lymphocytic leukemia (CLL), chronic myelogenous leukemia (cml), chronic myeloproliferative neoplasms, colorectal cancer, childhood colorectal cancer, craniopharyngioma (childhood brain cancer), cutaneous t-cell lymphoma, ductal carcinoma in situ (DCIS), embryonal tumors, (childhood brain CNS cancers), endometrial cancer (uterine cancer), ependymoma, esophageal cancer, childhood esophageal cancer, esthesioneuroblastoma (head and neck cancer), Ewing sarcoma (bone cancer), extracranial germ cell tumors, extragonadal germ cell tumors, eye cancer, childhood intraocular melanoma, intraocular melanoma, retinoblastoma, fallopian tube cancer, fibrous histiocytoma of bone (malignant, and osteosarcoma), gallbladder cancer, gastric (stomach) cancer, childhood gastric (stomach) cancer, gastrointestinal carcinoid tumor, gastrointestinal stromal tumors (gist) (soft tissue sarcoma), childhood gastrointestinal stromal tumors, germ cell tumors, childhood central nervous system germ cell tumors, childhood extracranial germ cell tumors, extragonadal germ cell tumors, ovarian germ cell tumors, testicular cancer, gestational trophoblastic disease, hairy cell leukemia, head and neck cancer, heart tumors, hepatocellular (liver) cancer, histiocytosis (Langerhans cell cancer), Hodgkin lymphoma, hypopharyngeal cancer (head and neck cancer), intraocular melanoma, childhood intraocular melanoma, islet cell tumors, (pancreatic neuroendocrine tumors), Kaposi sarcoma (soft tissue sarcoma), kidney (renal cell) cancer, Langerhans cell histiocytosis, laryngeal cancer (head and neck cancer), leukemia, lip and oral cavity cancer (head and neck cancer), liver cancer, lung cancer (non-small cell and small cell), childhood lung cancer, lymphoma, male breast cancer, malignant fibrous histiocytoma of bone and osteosarcoma, melanoma, childhood melanoma, melanoma (intraocular eye), childhood intraocular melanoma, Merkel cell carcinoma (skin cancer), mesothelioma, childhood mesothelioma, metastatic cancer, metastatic squamous neck cancer with occult primary (head and neck cancer), midline tract carcinoma with nut gene changes, mouth cancer (head and neck cancer), multiple endocrine neoplasia syndromes—see unusual cancers of childhood, multiple myeloma/plasma cell neoplasms, mycosis fungoides (lymphoma), myelodysplastic syndromes, myelodysplastic/myeloproliferative neoplasms, myelogenous leukemia, chronic (CML), myeloid leukemia, (acute AML), myeloproliferative neoplasms, nasal cavity and paranasal sinus cancer (head and neck cancer), nasopharyngeal cancer (head and neck cancer), neuroblastoma, non-hodgkin lymphoma, non-small cell lung cancer, oral cancer (lip and oral cavity cancer and oropharyngeal cancer), osteosarcoma and malignant fibrous histiocytoma of bone, ovarian cancer, childhood ovarian cancer, pancreatic cancer, childhood pancreatic cancer, pancreatic neuroendocrine tumors (islet cell tumors), papillomatosis, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood rhabdomyosarcoma (soft tissue sarcoma), childhood vascular tumors (soft tissue sarcoma), Ewing sarcoma (bone cancer), Kaposi sarcoma (soft tissue sarcoma), osteosarcoma (bone cancer), soft tissue sarcoma, uterine sarcoma, Sézary syndrome (lymphoma), skin cancer, childhood skin cancer, small cell lung cancer, small intestine cancer, soft tissue sarcoma, squamous cell carcinoma of the skin, squamous neck cancer with occult primary, stomach (gastric) cancer, childhood stomach, t-cell lymphoma, testicular cancer, childhood testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter kidney (renal cell cancer), ureter and renal pelvis (transitional cell cancer kidney renal cell cancer), urethral cancer, uterine cancer (endometrial), uterine sarcoma, vaginal cancer, childhood vaginal cancer, vascular tumors (soft tissue sarcoma), vulvar cancer, and Wilms tumor (and other childhood kidney tumors).

Urinary and Renal Stones and Related Disorders

Kidney stones (renal lithiasis, nephrolithiasis) are common in humans and animals, and they typically comprise hard deposits made of minerals and salts that form inside the bladder, kidneys, and urinary tract. Such stones often form when the urine becomes concentrated, allowing minerals to crystallize and stick together. Also, when a subject does not drink sufficient water there can be an accumulation of uric acid that is believed to be correlated with the formation of such stones. An excessively acidic environment in the urine of a subject is also thought to lead to the formation of kidney stones. They can be quite painful and can lead to complications such as the blocking of the tube connecting the kidney to the bladder. Embodiments of a transdermal delivery formulation provided herein have been found to be useful for the treatment, inhibit, amelioration of urinary and renal stones in a subject.

Accordingly, other embodiments provided herein are directed to methods of urinary and renal stones and related disorders. In an exemplary embodiment, a method of ameliorating or treating a urinary stone in accordance with the invention typically comprises topically and/or transdermally administering an effective amount of a transdermal delivery formulation comprising one or more buffering agent to a patient having a urinary stone and in need thereof, wherein said administration is effective to ameliorate, treat or reduce the symptoms of the urinary stone in said patient.

Examples of such conditions involving stones include, but not limited to bladder stones, kidney stones (calcium, calcium oxalate, calcium phosphate, cystine, magnesium ammonium phosphate, uric acid, struvite), renal stones, bilateral stone disease, urolithiasis during pregnancy, pediatric stones, stones in animals (e.g. urinary stones in animals), stones in patients with solitary kidneys, nephrolithiasis, other types of stones (e.g. bladder, urinary), patients with bleeding diathesis and related disorders, urolithiasis, as well as in conjunction with medical or surgical procedures such as a lithotripsy or ureteroscopy.

In certain embodiments, the patient is an animal such as a pet (e.g. cat, dog, bird), farm animal, or livestock. In non-limiting preferred embodiments, the urinary stone that is treated can be a bladder or kidney stone.

Skin Disorders

Other embodiments are directed to methods of treating a skin condition or disorder in a patient. These embodiments typically comprise topically and/or transdermally administering an effective amount of a transdermal delivery formulation comprising one or more buffering agent to a patient having a skin condition or disorder and in need thereof, wherein said administration is effective to ameliorate, treat or reduce the symptoms of the skin condition or disorder.

An exemplary but non-limiting skin disorder that is treated herein in particular embodiments is melasma. Melasma is a common skin problem that leads to skin pigmentation problems such as brown to gray-brown patches, usually on the face, cheeks, bridge of their nose, forehead, chin, and above their upper lip.

Melasma is believed to be triggered or worsened by birth control pills, pregnancy, and hormone therapy, stress, thyroid disease, and sun exposure. Sun exposure is believed to cause melasma because ultraviolet rays affect the cells that control pigment (melanocytes).

Thus, in certain embodiments methods of treating melasma are provided that comprise topically and/or transdermally administering an effective amount of a transdermal delivery formulation comprising one or more buffering agent to a patient having melasma and in need thereof, wherein said administration is effective to ameliorate, treat or reduce the symptoms of the melasma. In some embodiments, methods of the invention use a transdermal delivery formulation provided herein in conjunction with or co-administered with another treatment for melasma (e.g. sun protection or a sunscreen).

Another disorder or condition of the skin that is treated is skin damage. These embodiments typically comprise topically and/or transdermally administering an effective amount of a formulation comprising one or more buffering agent to a patient having skin damage and in need thereof, wherein said administration is effective to ameliorate, treat or reduce the skin damage or symptoms associated with the skin damage.

Other embodiments are directed to rejuvenating skin, and accordingly methods of rejuvenating skin are provided that comprise topically and/or transdermally administering an effective amount of a transdermal delivery formulation comprising one or more buffering agent to a subject in need of skin rejuvenation.

In certain embodiments, methods are provided that prevent or ameliorate collagen acylation in the skin of a patient. Alternative embodiments are also directed to the pre-treatment of skin to prevent or ameliorate skin damage caused by collagen acylation and other factors.

Another approach to make electrolyte balancing formulations is to avoid electrolyte imbalances by incorporating different buffers in different amount or ratios. Non-limiting examples of buffering agents that can be used together in different amounts or ratios include potassium bicarbonate, sodium bicarbonate, calcium carbonate, magnesium carbonate, and potassium carbonate. Mixtures of particular buffering agents including 2, 3, 4, 5, or more buffering agents are used depending on the formulation. Further, the relative amounts or ratio of each buffering agent may vary, for example, where the relative amounts are from 1:1.10 w/w; 1:1.15 w/w; 1:1.20 w/w; 1:1.25 w/w; 1:1.30 w/w; 1:1.35 w/w; 1:1.40 w/w; 1:1.45 w/w; 1:1.50 w/w; 1:1.55 w/w; 1:1.60 w/w; 1:1.65 w/w; 1:1.70 w/w; 1:1.75 w/w; 1:1.80 w/w; 1:1.85 w/w; 1:1.90 w/w; 1:1.95 w/w; 1:2 w/w; 1:2.5 w/w; 1:3 w/w; 1:3.5 w/w; 1:4 w/w, 1:4.5 w/w; 1:5 w/w, 1:5.5 w/w; 1:6 w/w; 1:6.5 w/w; 1:7 w/w; 1:8 w/w; 1:9 w/w; or 1:10 w/w. These ratios of buffering agents are applicable when two buffering agents are present, or more than two and the ratios are applicable between any two buffering agents.

Formulations

A formulation for transdermal delivery may, for example, comprise two components or it may comprise one or more buffering agent and a penetrant. Typically, however, a penetrant is less than 85% w/w. A transdermal delivery formulation may have a detergent of at least 1% w/w. For example, a suitable formulation may comprise about 10-56% w/w buffering agent and a penetrant. In one aspect, disclosed herein is a transdermal delivery formulation for transdermal delivery of one or more buffering agent through the skin of a subject, comprising: a buffering agent comprising a carbonate salt in an amount between about 10-56% w/w; a transdermal delivery formulation in an amount between about 5 to 55% w/w; a detergent portion in an amount of at least 1% w/w; and wherein the formulation comprises water in an amount from none up to about 77% w/w.

In an embodiment, a carbonate, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In another aspect, disclosed herein is a method for transdermal delivery of a carbonate salt of a %, at least comprising: a buffering agent comprising a carbonate salt in an amount between about 10-45% w/w; a transdermal delivery formulation in an amount between about 5 to 55% w/w; a detergent portion in an amount between about 1 to 15% w/w; and wherein the formulation comprises water in an amount between about 15 to 65% w/w, through the skin of a subject, wherein the carbonate salt of the formulation is in an amount between about 15-32% w/w of the formulation.

In another embodiment, a buffering agent comprising a carbonate salt, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In yet another aspect, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount between about 10-45% w/w; a transdermal delivery formulation in an amount between about 5 to 55% w/w; a detergent portion in an amount between about 1 to 15% w/w; wherein the formulation comprises water in an amount between about 15 to 65% w/w, through the skin of a subject, wherein the carbonate salt of the formulation is in an amount between about 15-32% w/w of the formulation, therapeutic, and wherein the alkalinity of the formulation enhances penetration of the therapeutic agent.

In one aspect, disclosed herein is a formulation for transdermal delivery of one or more buffering agent through the skin of a subject, comprising: a buffering agent comprising a carbonate salt in an amount between about 10-45% w/w; a transdermal delivery formulation in an amount between about 5 to 55% w/w; a detergent portion in an amount between about 1 to 15% w/w; and wherein the formulation comprises water in an amount between about 15 to 65% w/w, and wherein the formulation comprises less than about 12% w/w of the transdermal delivery formulation.

In another aspect, disclosed herein is a method for transdermal delivery of a carbonate salt of the formulation comprising: a buffering agent comprising a carbonate salt in an amount between about 10-45% w/w; a transdermal delivery formulation in an amount between about 5 to 55% w/w; a detergent portion in an amount between about 1 to 15% w/w; and wherein the formulation comprises water in an amount between about 15 to 65% w/w, and wherein the formulation comprises less than about 12% w/w of the transdermal delivery formulation, through the skin of a subject, wherein the carbonate salt of the formulation is in an amount between about 15-32% w/w of the formulation, wherein the formulation comprises less than about 12% w/w of the transdermal delivery formulation, and wherein the alkalinity of the formulation enhances penetration of the therapeutic agent.

In yet another aspect, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount between about 10-45% w/w; a transdermal delivery formulation in an amount between about 5 to 55% w/w; a detergent portion in an amount between about 1 to 15% w/w; wherein the formulation comprises water in an amount between about 15 to 65% w/w, through the skin of a subject, wherein the carbonate salt of the formulation is in an amount between about 15-32% w/w of the formulation, and wherein the formulation comprises less than about 12% w/w of the transdermal delivery formulation.

In some embodiments, a suitable transdermal delivery formulation comprises: Siligel™ in an amount less than about 5% w/w; water in an amount between about 10-65% w/w; isopropyl palmitate in an amount between about 0.5-10% w/w; stearic Acid in an amount between about 0.25-10% w/w; cetyl alcohol in an amount between about 0.25-10% w/w; glycerin in an amount between about 0.25-5% w/w; a transdermal delivery formulation in an amount between about 0.25-10% w/w; ethanol in an amount less than about 5% w/w; benzyl alcohol in an amount less than about 5% w/w; sodium hydroxide 50% w/v in an amount between about 0.1-5% w/w; and sodium bicarbonate in an amount between about 1-32% w/w.

In some embodiments, a suitable transdermal delivery formulation comprises: Aveeno® in an amount between about 20-85% w/w; and sodium bicarbonate (3DF) in an amount between about 15-45% w/w.

In some embodiments, a transdermal delivery formulation comprises: Aveeno® in an amount between about 20-85% w/w; and sodium bicarbonate (Milled #7) in an amount between about 15-45% w/w.

In some embodiments, a suitable transdermal delivery formulation comprises: Siligel™ in an amount less than about 5% w/w; water in an amount between about 10-55% w/w; isopropyl palmitate in an amount between about 0.5-10% w/w; stearic Acid in an amount between about 0.25-5% w/w; Cetyl alcohol in an amount between about 0.25-10% w/w; almond oil in an amount between about 0.5-10% w/w; propylene glycol in an amount between about 0.25-10% w/w; ethanol in an amount less than about 5% w/w; benzyl alcohol in an amount less than about 5% w/w; sodium hydroxide 50% w/v in an amount between about 0.1-5% w/w; and sodium bicarbonate in an amount between about 1-32% w/w.

The surprising effects achieved by the formulations and methods of the present invention are in part attributable to an improved transdermal delivery formulation that enhances delivery of a carbonate salt through the skin. The present transdermal delivery formulations may include a nonionic surfactant. Applicant has found that by employing carbonate salts with particle sizes as disclosed herein, delivered with the penetrants as disclosed herein, and in some embodiments providing a combination of a nonionic surfactant and a polar gelling agent, the penetration capabilities of the carbonate salts of the resulting formulation and the effective level of delivery of the carbonate salts has been enhanced.

In a transdermal delivery formulation, penetrants are based on combinations of an alcohol, such as benzyl alcohol to provide a concentration of 0.5-20% w/w of the final formulation with a transdermal delivery formulation present to provide 25-70% w/w of the formulation. These penetrants are also useful when the agent is a buffer, such as sodium bicarbonate, but less of a transdermal delivery formulation may be required—e.g. less than 12% w/w when the sodium bicarbonate is present at high concentration as disclosed herein.

In some embodiments, the buffering component is any mildly basic compound or combination that will result in a pH of 7-8 in the microenvironment of the tumor cells. In some embodiments, the formulation has a pH of 7-10. Such buffers, in addition to carbonate and/or bicarbonate salts, include lysine buffers, chloroacetate buffers, tris buffers (i.e., buffers employing tris (hydroxymethyl) aminoethane), phosphate buffers and buffers employing non-natural amino acids with similar pKa values to lysine. In some embodiments, the carbonate and/or bicarbonate salt is in an amount between about 7-32% w/w of the formulation. For example, the enantiomers of native forms of such amino acids or analogs of lysine with longer or shorter carbon chains or branched forms thereof. Histidine buffers may also be used. Typically, the concentration of buffer in the compositions is in the range of 10-50% w/w. More typical ranges for sodium bicarbonate or sodium carbonate or both are 10-35% w/w. In some embodiments, the carbonate salt is in an amount between about 15-32% w/w of the formulation.

Alternatively, the penetrant component comprises a completion component as well as one or more electrolytes sufficient to impart viscosity and viscoelasticity, one or more surfactants and an alcohol. The completion component can be a polar liquid, a non-polar liquid or an amphiphilic substance.

The percentage of carbonate salt in a transdermal delivery formulation will depend upon the amount required to be delivered in order to have a useful effect on treating the disorder. In general, the carbonate salt may be present in the formulation in an amount as low as 1% w/w up to about 50% w/w. Typical concentrations may include 15-32% w/w. Since the required percentage of carbonate salt depends on the frequency of administration, as well as the time allotted for administration for each application, the level of carbonate salt may be varied over a wide range. In some embodiments, the carbonate salt is sodium carbonate and/or sodium bicarbonate milled to a particle size is less than 200 μm. In some embodiments, the carbonate salt is sodium carbonate and/or sodium bicarbonate milled to a particle size is less than 70 μm. In some embodiments, the carbonate salt is sodium carbonate and/or sodium bicarbonate milled to a particle size is less than 70 μm, wherein the sodium bicarbonate is solubilized in the formulation in an amount less than 20% w/w of a transdermal delivery formulation. In some embodiments, the carbonate salt is sodium carbonate and/or sodium bicarbonate milled to a particle size is less than 70 μm, wherein particle sizes less than about 10 μm have an enhanced penetration thru the skin of a subject. In some embodiments, the sodium carbonate and/or sodium bicarbonate are jet milled to a particle size less than about 70 μm. In some embodiments, the sodium bicarbonate is Sodium Bicarbonate USP Grade 3DF that has a particle size distribution less than 70 μm.

A transdermal delivery formulation of the disclosure may be prepared in a number of ways. Typically, the components of a transdermal delivery formulation are simply mixed together in the required amounts. However, it is also desirable in some instances to, for example, carry out dissolution of a carbonate salt and then add a separate preparation containing the components aiding the delivery of the carbonate salts in the form of a carrier. The concentrations of these components in the carrier, then, will be somewhat higher than the concentrations required in a final transdermal delivery formulation. Thus, sodium bicarbonate may first be dissolved in water and then added to a carrier comprising an alcohol, a transdermal delivery formulation and optionally a combination of a nonionic surfactant and polar gelling agent, or of ionic detergent. Alternatively, some subset of these components can first be mixed and then “topped off” with the remaining components either simultaneously or sequentially. The precise manner of preparing a transdermal delivery formulation will depend on the choice of carbonates and the percentages of the remaining components that are desirable with respect to that carbonate salt. In some embodiments, the water is in an amount between about 10-85% w/w, 15-50% w/w, or 15-45% w/w of the formulation.

The transdermal delivery formulation is a multi-component mixture, whereby the particular concentrations of the penetration enhancers are informed in part by the molecular mass of the sodium bicarbonate, or sodium bicarbonate and the therapeutic agent to be transported. A transdermal delivery formulation enables the sodium bicarbonate and/or therapeutic agent to become bio-available to the target site within minutes of topical administration. A transdermal delivery formulation permit the use of minimal concentrations of therapeutic agents, as little as. 1/1000th of concentrations required of alternative processes, while enabling bioactivity and positive clinical outcomes simultaneously. In some embodiments, the transdermal delivery formulation comprises an alcohol in an amount less than 5% w/w of the formulation.

Administration and Dosing

A transdermal delivery formulation provided herein can be topically administered in any form. For administration for the treatment of skin conditions a sufficient amount of the topical composition can be applied onto a desired area and surrounding skin, for example, in an amount sufficient to cover a desired skin surface. A transdermal delivery formulation can be applied to any skin surface, including for example, facial skin, and the skin of the hands, neck, chest and/or scalp.

In applying a transdermal delivery formulation of the invention, a transdermal delivery formulation itself is simply placed on the skin and spread across the surface and/or massaged to aid in penetration. The amount of transdermal delivery formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a transdermal delivery formulation to the skin and prevents distortion of a transdermal delivery formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed transdermal delivery formulations that penetrate intact skin may be applied using occlusive patches, such as those in the form micro-patches. External reservoirs of the formulations for extended administration may also be employed.

Accordingly, in certain embodiments alternative methods of administering one or more buffering agent, therapeutic compounds, agents, drugs through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

It has been found, generally, that the requirements for effective penetration of the skin in the case of buffers as active agents are less restrictive than those required for alternative agents useful in preventing cancer metastasis. In addition, although for these indications' delivery to the locus of the solid tumor, including melanoma, or melasma or gout is desirable, effective systemic pH alteration can be used as a way to diagnose the effectiveness of penetration when topical administration is employed.

The application method is determined by the nature of the treatment but may be less critical than the nature of a transdermal delivery formulation itself. If the application is to a skin area, it may be helpful in some instances to prepare the skin by cleansing or exfoliation. In some instances, it is helpful to adjust the pH of the skin area prior to application of the formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In some embodiments, the disclosure is directed to administering a therapeutic agent in combination with a formulation or method provided herein. A wide variety of therapeutic agents may be used in a transdermal delivery formulation or compositions and formulations for other routes of administration, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply affect transport across the skin into a localized subdermal location, such as treatment of nail fungus or modulation of hair growth or may affect systemic delivery such as is desirable in some instances where vaccines are used.

For administration of anesthetics as the therapeutic agent, the local anesthetic may be one or more of the following: benzocaine, lidocaine, tetracaine, bupivacaine, cocaine, etidocaine, mepivacaine, pramoxine, prilocaine, procaine, chloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof. Combinations of anesthetic agents may also be used. The anesthetic agent{s) are included in the composition in effective amount(s). Depending on the anesthetic(s) the amounts of anesthetic or combination is typically in the range of 1% w/w to 50% w/w. The compositions of the invention provide rapid, penetrating relief that is long lasting. The pain to be treated can be either traumatic pain and/or inflammatory pain.

In addition to a transdermal delivery formulation of the invention per se, the methods may employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow carbonates to pass through the epidermis to the dermis at least, and, if systemic administration is achieved, through the dermis itself.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

A transdermal delivery formulation can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, for 4 weeks, or for 4 weeks or more. In some instances, it may also be desirable to continue treatment indefinitely for example to inhibit or prevent carcinogenesis or for improving, extending the duration of remission, or maintaining remission of a cancer or another disease or disorder. A suitable administration for a transdermal delivery formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

The present t transdermal delivery formulation remains stable in storage for periods including up to about 5 years, between about 3 months and about 5 years, between about 3 months and about 4 years, between about 3 months and about 3 years, and alternately any time period between about 6 months and about 3 years.

A transdermal delivery formulation described herein remains stable for up to at least 3 years at a temperature of less than or equal to 40° C. In an embodiment, the presently described transdermal delivery formulation remains stable for at least 2 years at a temperature of less than or equal to 40° C. In an embodiment, the presently described transdermal delivery formulation remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described transdermal delivery formulation in accordance with the subject matter described herein remains stable for an extended period of time when packaged in a multi-use container such as a bottle dispenser or the like, and exhibits equal to or even greater stability when packaged in a single-use package.

In another aspect, the transdermal delivery formulation of certain embodiments comprises a daily dose of particular buffering compound (e.g. sodium bicarbonate, sodium carbonate, magnesium carbonate, potassium carbonate, potassium bicarbonate, TRIS, Lysine, IEPA, etc.). A daily dose for topical or transdermal administration of a transdermal delivery formulation depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

Alternatively, a suitable daily dose for a transdermal delivery formulation of each of one or more particular buffering compound (e.g. sodium bicarbonate, sodium carbonate, magnesium carbonate, potassium carbonate, potassium bicarbonate, TRIS, Lysine, IEPA, etc.) is at least about 1 mg/kg, at least about 10 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, at least about 500 mg/kg, at least about 550 mg/kg, at least about 600 mg/kg, at least about 700 mg/kg, at least about 800 mg/kg, at least about 900 mg/kg, at least about 1 g/kg, at least about 2 g/kg, at least about 3 g/kg, or at least about 5 g/kg.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

The present transdermal delivery formulation remains stable in storage for periods including up to about 5 years, between about 3 months and about 5 years, between about 3 months and about 4 years, between about 3 months and about 3 years, and alternately any time period between about 6 months and about 3 years.

Alternatively, a suitable dose for topical or transdermal administration of each of one or more particular buffering compound (e.g. sodium bicarbonate, sodium carbonate, magnesium carbonate, potassium carbonate, potassium bicarbonate, TRIS, Lysine, IEPA, etc.) for subject is at least about 100 mg, at least about 500 mg, at least about 1 g, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This does may be administered daily, twice a day, three times a day, four times a day, five times a day, or more than five times a day.

Aspects of the present specification disclose that the symptoms associated with a disease or disorder described herein are reduced following application of a transdermal delivery formulation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced following application of a transdermal delivery formulation by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another aspect, in certain embodiments a pH modulating transdermal delivery formulation (e.g. containing sodium bicarbonate) is administered topically or transdermally such that the dose results in a subject intake of at least about 0.1 nmol/hr/Kg, at least about 0.5 nmol/hr/Kg, at least about 0.7 nmol/hr/Kg, at least about 1.0 nmol/hr/Kg, at least about 1.1 nmol/hr/Kg, at least about 1.2 nmol/hr/Kg, at least about 1.3 nmol/hr/Kg, at least about 1.4 nmol/hr/Kg, at least about 1.5 nmol/hr/Kg, at least about 1.6 nmol/hr/Kg, at least about 1.7 nmol/hr/Kg, at least about 1.8 nmol/hr/Kg, at least about 1.9 nmol/hr/Kg, at least about 2.0 nmol/hr/Kg, at least about 2.5 nmol/hr/Kg, at least about 3.0 nmol/hr/Kg, at least about 3.5 nmol/hr/Kg, at least about 4.0 nmol/hr/Kg, at least about 5 nmol/hr/Kg, at least about 10 nmol/hr/Kg, at least about 25 nmol/hr/Kg, at least about 50 nmol/hr/Kg, at least about 100 nmol/hr/Kg, at least about 500 nmol/hr/Kg, or at least about 1 μmol/hr/Kg,

In another aspect, in certain embodiments a pH modulating transdermal delivery formulation (e.g. containing sodium bicarbonate) is administered topically or transdermally such that the dose results in a peak plasma concentration of a buffering or pH modulating compound ranges from about 1 μg/ml to 50 μg/ml, about 5 μg/ml to about 45 μg/ml, about 5 μg/ml to about 40 μg/ml, about 5 μg/ml to about 35 μg/ml, about 5 μg/ml to about 30 μg/ml, about 5 μg/ml to about 25 μg/ml, about 1 μg/ml to about 45 μg/ml, about 1 μg/ml to about 40 μg/ml, about 1 μg/ml to about 35 μg/ml, about 1 μg/ml to about 30 μg/ml, about 1 μg/ml to about 25 μg/ml, about 1 μg/ml to about 20 μg/ml, about 1 μg/ml to about 15 μg/ml, about 1 μg/ml to about 10 μg/ml, about 1 μg/ml to about 9 μg/ml, about 1 μg/ml to about 8 μg/ml, about 1 μg/ml to about 7 μg/ml, about 1 μg/ml to about 6 μg/ml, and about 1 μg/ml to about 5 μg/ml.

In another aspect, in certain embodiments a pH modulating transdermal delivery formulation (e.g. containing sodium bicarbonate) is administered topically or transdermally so that plasma concentration ranges from about 1 ng/ml to 500 μg/ml, about 10 ng/ml to 500 μg/ml, about 100 ng/ml to 500 μg/ml, about 1 μg/ml to 500 μg/ml, about 10 μg/ml to 500 μg/ml, about 25 μg/ml to 500 μg/ml, about 25 μg/ml to about 450 μg/ml, about 25 μg/ml to about 400 μg/ml, about 25 μg/ml to about 350 μg/ml, about 25 μg/ml to about 300 μg/ml, about 25 μg/ml to about 250 μg/ml, about 50 μg/ml to about 500 μg/ml, about 55 μg/ml to about 500 μg/ml, about 60 μg/ml to about 500 μg/ml, about 65 μg/ml to about 500 μg/ml, about 70 μg/ml to about 500 μg/ml, about 75 μg/ml to about 500 μg/ml, about 80 μg/ml to about 500 μg/ml, about 85 μg/ml to about 500 μg/ml, about 90 μg/ml to about 500 μg/ml, about 95 μg/ml to about 500 μg/ml, about 100 μg/ml to about 500 μg/ml, about 110 μg/ml to about 500 μg/ml, about 120 μg/ml to about 500 μg/ml, about 130 μg/ml to about 500 μg/ml, about 140 μg/ml to about 500 μg/ml about 150 μg/ml to about 500 μg/ml, about 160 μg/ml to about 500 μg/ml, about 170 μg/ml to about 500 μg/ml, about 180 μg/ml to about 500 μg/ml, about 200 μg/ml to about 500 μg/ml, about 200 μg/ml to about 490 μg/ml, about 200 μg/ml to about 480 μg/ml, about 200 μg/ml to about 470 μg/ml, about 200 μg/ml to about 460 μg/ml, about 200 μg/ml to about 450 μg/ml, about 200 μg/ml to about 440 μg/ml, about 200 μg/ml to about 430 μg/ml, or about 200 μg/ml to about 400 μg/ml.

In further embodiments, a pH modulating transdermal delivery formulation (e.g. containing sodium bicarbonate) is administered topically or transdermally so that plasma concentration is at least 10 ng/ml, at least 25 ng/ml, at least 50 ng/ml, at least 100 ng/ml, at least 250 ng/ml, at least 0.5 μg/ml, at least 0.75 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml or more than 100 μg/ml.

In another aspect, a pH modulating transdermal delivery formulation (e.g. containing sodium bicarbonate) is administered topically or transdermally so that peak plasma concentration is reached in 10 min, 15 min, 20 min, 30 min, 45 min, 60 min, 75 min, 90 min, 2 hr, 3 hr, 4 hr, 5 hr, 6 hr, 7 hr, 8 hr, 10 hr, 12 hr or 24 hr after administration.

Aspects of the present specification disclose that the symptoms associated with a disease or disorder described herein are reduced following administration of a transdermal delivery formulation of the present invention by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A transdermal delivery formulation as described herein can be used in the manufacture of medicaments and for the treatment of humans and other animals by administration in accordance with conventional procedures.

Dosing can be single dosage or cumulative (serial dosing), and can be readily determined by one skilled in the art. A transdermal delivery formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a transdermal delivery formulation as disclosed herein. Alternatively, treatment of a disease may comprise multiple administrations of an effective dose of a transdermal delivery formulation as carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a transdermal delivery formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a transdermal delivery formulation disclosed herein that is administered can be adjusted accordingly. In one embodiment, a transdermal delivery formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In one embodiment, an anti-cancer transdermal delivery formulation disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an anti-cancer transdermal delivery formulation is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, an anti-cancer transdermal delivery formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of an anti-cancer transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of an anti-cancer transdermal delivery formulation disclosed herein reduces or maintains a cancer cell population and/or tumor cell size in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of an anti-cancer transdermal delivery formulation disclosed herein reduces or maintains a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of an anti-cancer transdermal delivery formulation disclosed herein reduces or maintains a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A transdermal delivery formulation disclosed herein may comprise an ati=cancer transdermal delivery formulation in a therapeutically effective amount. As used herein, the term “effective amount” is synonymous with “therapeutically effective amount”, “effective dose”, or “therapeutically effective dose” and when used in reference to reducing or maintaining a cancer cell population and/or tumor cell size in an individual refers to the minimum dose of a cancer therapeutic disclosed herein necessary to achieve the desired therapeutic effect and includes a dose sufficient to reduce or maintain of cancer cell population and/or tumor cell size in an individual. The effectiveness of an anti-cancer transdermal delivery formulation disclosed herein capable of reducing or maintaining a cancer cell population and/or tumor cell size in an individual can be determined by observing an improvement in an individual based upon one or more clinical symptoms, and/or physiological indicators associated with reducing or maintaining a cancer cell population and/or tumor cell size in an individual. Maintenance or a reduction of cancer cell population and/or tumor cell size can be indicated by a reduced need for a concurrent therapy. The effectiveness of an anti-cancer transdermal delivery formulation disclosed herein capable of reducing or maintaining a cancer cell population and/or tumor cell size in an individual can be determined by observing an improvement in an individual based upon one or more clinical symptoms, and/or physiological indicators associated with a reduction or maintenance of cancer cell population and/or tumor cell size. The effectiveness of an anti-cancer transdermal delivery formulation disclosed herein is also capable of prolonging the life of an individual as compared to the same individual if the anti-cancer transdermal delivery formulation is not administered. The effectiveness of anti-cancer transdermal delivery formulation disclosed herein is also capable of enhancing the quality of life of an individual as compared to the same individual if the anti-cancer transdermal delivery formulation is not administered.

The appropriate effective amount of an anti-cancer transdermal delivery formulation disclosed herein to be administered to reduce or maintain of a cancer cell population and/or tumor cell size in an individual condition can be determined by a person of ordinary skill in the art by taking into account factors, including, without limitation, the measured number of cancer cells in blood samples or biopsies or CAT scans, PET scans, NMR and/or sonagrams taken from or of the individual, the particular characteristics, history and risk factors of the patient, such as, e.g., age, weight, general health and the like, or any combination thereof. Additionally, where repeated administration of an anti-cancer transdermal delivery formulation is used, an effective amount of an anti-cancer transdermal delivery formulation will further depend upon factors, including, without limitation, the frequency of administration, the half-life of the anti-cancer transdermal delivery formulation, or any combination thereof. In is known by a person of ordinary skill in the art that an effective amount of an anti-cancer transdermal delivery formulation disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of an anti-cancer transdermal delivery formulation disclosed herein generally would be expected to require higher dosage levels than administration by inhalation. Similarly, systemic administration of an anti-cancer transdermal delivery formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a cancer therapeutic disclosed herein that is administered can be adjusted accordingly.

Aspects of the present specification disclose, in part, reduction or maintenance of cancer cell population and/or tumor cell size in an individual. As used herein, the term “treating,” refers to reduction or maintenance of cancer cell population and/or tumor cell size in an individual. For example, the term “treating” can mean reduction or maintenance of cancer cell population and/or tumor cell size levels in an individual by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with cancer, including the detection of cancer cell population and/or tumor cell size are well known and can be determined by a person of ordinary skill in the art by using commonly known testing means, including blood tests, CT scans sonagrams and other tests known to those of ordinary skill. Those of skill in the art will know the appropriate symptoms or indicators associated with cancer and will know how to determine if an individual is a candidate for treatment as disclosed herein.

In an embodiment, a first anti-cancer transdermal delivery formulation is administered to an individual and at a later date, a second anti-cancer transdermal delivery formulation is administered to the same individual. In an embodiment, a first anti-cancer transdermal delivery formulation is administered to an individual at the same time as a second anti-cancer transdermal delivery formulation is administered to the individual.

A transdermal delivery formulation as disclosed herein is administered to an individual. An individual is typically a human being, but can be an animal, including, but not limited to, dogs, cats, birds, cattle, horses, sheep, goats, reptiles and other animals, whether domesticated or not.

In one aspect, disclosed herein is a formulation for transdermal delivery of an active agent through the skin, nail or hair follicle of a subject, wherein the formulation comprises a) a transdermal delivery formulation in an amount less than about 60% w/w, comprising i. one or more phosphatides and ii. one or more fatty acids; and b) water in an amount less than about 50% w/w.

In some embodiments, the formulation comprises a) a transdermal delivery formulation in an amount less than about 60% w/w, comprising i. one or more phosphatides and ii. one or more fatty acids; and b) water in an amount less than about 50% w/w, further comprises benzyl alcohol in an amount between about 0.5-5% w/w.

In some embodiments, the transdermal delivery formulation comprises benzyl alcohol in an amount less than 5% w/w of the formulation.

In some embodiments, the formulation comprises a) a transdermal delivery formulation in an amount less than about 60% w/w, comprising i. one or more phosphatides and ii. one or more fatty acids; and b) water in an amount less than about 50% w/w, further comprises Isopropyl Palmitate in an amount between about 5-5% w/w.

In some embodiments, the water is deionized water and/or purified water.

In some embodiments, the water is deionized water and/or purified water.

In some embodiments, the water is in an amount between about 15-40% w/w of the formulation.

In some embodiments, the one or more phosphatides in an amount between about 0.5-55% w/w of the transdermal delivery formulation.

In some embodiments, the transdermal delivery formulation comprises phosphatidylcholine, hydrogenated phosphatidylcholine, phosphatidylserine, phosphatidylethanolamine, phosphatidylinositol, or a combination thereof in amount less than 30% w/w of the formulation.

In some embodiments, the one or more phosphatides comprises phosphatidylcholine of the transdermal delivery formulation.

In some embodiments, the one or more fatty acids in an amount between about 1-35% w/w of the transdermal delivery formulation.

In some embodiments, the one or more fatty acids in an amount between about 5-35% w/w of the transdermal delivery formulation.

In some embodiments, the one or more fatty acids comprises Linoleic Acid, Oleic Acid, Stearic Acid, sunflower oil, or a combination thereof.

In some embodiments, the one or more fatty acids comprises Linoleic Acid.

In some embodiments, the one or more fatty acids comprises Oleic Acid.

In some embodiments, the one or more fatty acids comprises Stearic Acid.

In some embodiments, the one or more phosphatides are derived from a seed oil in an amount between about 0.5-55% w/w of the transdermal delivery formulation.

In some embodiments, the one or more phosphatides are derived from a seed oil in an amount between about 5-35% w/w of the transdermal delivery formulation.

In some embodiments, the one or more phosphatides are derived from a sunflower oil in an amount between about 0.5-55% w/w of the transdermal delivery formulation.

In some embodiments, the one or more phosphatides are derived from a sunflower oil in an amount between about 5-35% w/w of the transdermal delivery formulation.

In some embodiments, the one or more phosphatides are derived from an almond oil in an amount between about 0.5-55% w/w of the transdermal delivery formulation.

In some embodiments, the one or more phosphatides are derived from an almond oil in an amount between about 5-35% w/w of the transdermal delivery formulation.

In some embodiments, the one or more phosphatides comprises one or more fatty acids derived from soy lecithin.

In some embodiments, the glucose in an amount between about 0.05-10% w/w of the transdermal delivery formulation. In another embodiment, the transdermal delivery formulation contains no glucose.

In some embodiments, the glucose is anhydrous dextrose in an amount between about 0.05-10% w/w of the transdermal delivery formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a nonionic surfactant in an amount between about 2-25% w/w of the transdermal delivery formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a polar solvent at least in an amount in molar excess of the nonionic surfactant.

In some embodiments, the nonionic surfactant is a poloxamer and the polar solvent is water.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a polar solvent in an amount less than 5% w/w of the formulation.

In some embodiments, the transdermal delivery formulation further comprises a detergent portion in an amount between about 1-30% w/w of the transdermal delivery formulation.

In some embodiments, the detergent portion comprises a nonionic surfactant in an amount between about 2-25% w/w of the transdermal delivery formulation; and a polar solvent in an amount less than 5% w/w of the transdermal delivery formulation.

In some embodiments, the transdermal delivery formulation is in an amount between about 10-60% w/w of the transdermal delivery formulation.

In some embodiments, the transdermal delivery formulation comprises an alcohol in an amount less than 10% w/w of the transdermal delivery formulation.

In some embodiments, the transdermal delivery formulation further comprises an alcohol, a surfactant, and a polar solvent.

In some embodiments, the transdermal delivery formulation comprises cetyl alcohol in amount less than 5% w/w of the formulation.

In some embodiments, the transdermal delivery formulation comprises ethanol in an amount less than 5% w/w of the formulation.

In some embodiments, the transdermal delivery formulation comprises glycerine in an amount less than 5% w/w of the formulation.

In some embodiments, the transdermal delivery formulation comprises propylene glycol in an amount less than 8% w/w of the formulation.

In some embodiments, the formulation comprises a gelling agent in an amount less than 20% w/w of the formulation.

In some embodiments, the formulation comprises menthol in an amount between about 0.05-5% w/w of the formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises tranexamic acid in an amount less than 5% w/w of the formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a humectant, an emulsifier, an emollient, or a combination thereof.

In some embodiments, the formulation has a pH of 9-11.

In some embodiments, the formulation has a pH of 7-10.5.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises an active agent.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises an active agent component in an amount less than about 60% w/w.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises an active agent component in an amount less than about 60% w/w, wherein the active agent is an anesthetic, a fat-dissolving agent, one or more nutrients, a tissue volume enhancer, a vaccine component, a hair growth modulator, an antifungal agent, an agent to promote smoking cessation, a cannabinoid, Withaferin A, a buffering agent, a chemotherapeutic, an immunotherapeutic agent, one or more protease inhibitors, iron or one or more iron containing compounds, one or more ketone or ketone derived components, one or more dermal contouring agents, or a combination thereof.

In some embodiments, the buffering agent is sodium carbonate and/or sodium bicarbonate.

In some embodiments, the cannabinoid is a crystalline cannabidiol.

In another aspect disclosed herein is a method to effect transdermal delivery of an active ingredient comprising applying to the skin, nails or hair follicles of a subject an effective amount of the formulation comprising a) a transdermal delivery formulation in an amount less than about 60% w/w, comprising i. one or more phosphatides, ii. glucose, and iii. one or more fatty acids; and b) water in an amount less than about 50% w/w, further comprises an active agent.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof. Ultimately, the formulations may be utilized in virtually any context where buffering therapy with or without a therapeutic agent(s) is desired.

### Example 1

### Tumor Responsiveness Testing to Topical Buffering Agents and Proteases

In this experiment, tumor biopsy specimens are incubated in various formulations and mediums, including pH neutral mediums and alkaline mediums to determine responsiveness to buffer therapies.

Transdermal delivery formulations of the invention are tested in some studies for the ability to modify or reduce protein secretion or in other experiments to inhibit multiple stages of tumor progression with and without coadministration and coformulation of topically applied buffering agents in formulations of the invention.

One measurement in these experiments is to determine if tumor cells are sensitive to particular proteases and by altering their morphology or by acidifying their microenvironment when included in a transdermal formulation that is applied to a patient.

Accordingly, in another aspect a diagnostic test is provided for responsiveness of a patient or subject to one or more protease inhibitor as therapeutic agents. Additional diagnostic test provided herein examine responsiveness to one or more protease inhibitor administered in combination with a formulation comprising one or more buffering agent provided herein or formulated with a formulation comprising one or more buffering agent. Proteases inhibitors are administered alone or in combination with formulations comprising one or more buffering agent provided herein to determine if the tumor cells are pH sensitive and therefore may be more responsive if a buffering agent is included in the therapy.

1. A transdermal delivery formulation for transdermal delivery of an active agent through the skin, nail or hair follicle of a subject, wherein the transdermal delivery formulation comprises:

a) a transdermal delivery formulation in an amount less than about 60% w/w, comprising:
i. one or more phosphatides,
ii. glucose, and
iii. one or more fatty acids; and

b) water in an amount less than about 50% w/w.

2. The transdermal delivery formulation of claim 1, further comprising benzyl alcohol in an amount between about 0.5-5% w/w.
3. The transdermal delivery formulation of claim 1 wherein the transdermal delivery formulation comprises benzyl alcohol in an amount less than 5% w/w of the formulation.
4. The transdermal delivery formulation of claim 1, further comprising Isopropyl Palmitate in an amount between about 5-5% w/w.
5. The transdermal delivery formulation of claim 1, wherein the water is deionized water and/or purified water.
6. The transdermal delivery formulation of claim 1, wherein the water is not deionized water and/or purified water.
7. The transdermal delivery formulation of claim 1, wherein the water is in an amount between about 15-40% w/w of the formulation.
8. The formulation of claim 1, wherein the one or more phosphatides in an amount between about 0.5-55% w/w of the transdermal delivery formulation.
9. The transdermal delivery formulation of claim 1, wherein the transdermal delivery formulation comprises phosphatidylcholine, hydrogenated phosphatidylcholine, phosphatidylserine, phosphatidylethanolamine, phosphatidylinositol, or a combination thereof in amount less than 30% w/w of the formulation.
10. The transdermal delivery formulation of claim 1, wherein the one or more phosphatides comprises phosphatidylcholine of the transdermal delivery formulation.
11. The transdermal delivery formulation of claim 1, wherein the one or more fatty acids in an amount between about 1-35% w/w of the transdermal delivery formulation.
12. The transdermal delivery formulation of claim 1, wherein the one or more fatty acids in an amount between about 5-35% w/w of the transdermal delivery formulation.
13. The transdermal delivery formulation of claim 1, wherein the one or more fatty acids comprises Linoleic Acid, Oleic Acid, Stearic Acid, safflower oil, or a combination thereof.
14. The transdermal delivery formulation of claim 1, wherein the one or more fatty acids comprises Linoleic Acid.
15. The transdermal delivery formulation of claim 1, wherein the one or more fatty acids comprises Oleic Acid.
16. The transdermal delivery formulation of claim 1, wherein the one or more fatty acids comprises Stearic Acid.
17. The transdermal delivery formulation of claim 1, wherein the one or more fatty acids are derived from a seed oil in an amount between about 0.5-55% w/w of the transdermal delivery formulation.
18. The transdermal delivery formulation of claim 1, wherein the one or more fatty acids are derived from a seed oil in an amount between about 5-35% w/w of the transdermal delivery formulation.
19. The transdermal delivery formulation of claim 1, wherein the one or more fatty acids are derived from a safflower oil in an amount between about 0.5-55% w/w of the transdermal delivery formulation.
20. The transdermal delivery formulation of claim 1, wherein the one or more fatty acids are derived from a safflower oil in an amount between about 5-35% w/w of the transdermal delivery formulation.
21. The transdermal delivery formulation of claim 1, wherein the one or more fatty acids are derived from an almond oil in an amount between about 0.5-55% w/w of the transdermal delivery formulation.
22. The transdermal delivery formulation of claim 1, wherein the one or more fatty acids are derived from an almond oil in an amount between about 5-35% w/w of the transdermal delivery formulation.
23. The transdermal delivery formulation of claim 1, wherein the one or more fatty acids comprises one or more fatty acids derived from soy lecithin.
24. The transdermal delivery formulation of claim 1, wherein the glucose in an amount between about 0.05-10% w/w of the transdermal delivery formulation.
25. The transdermal delivery formulation of claim 1, wherein the glucose is anhydrous dextrose in an amount between about 0.05-10% w/w of the transdermal delivery formulation.
26. The transdermal delivery formulation of claim 1, further comprises a nonionic surfactant in an amount between about 2-25% w/w of the transdermal delivery formulation.
27. The transdermal delivery formulation of claim 26, further comprises a polar solvent at least in an amount in molar excess of the nonionic surfactant.
28. The transdermal delivery formulation of claim 27, wherein the nonionic surfactant is a poloxamer and the polar solvent is water.
29. The transdermal delivery formulation of claim 1, further comprising a polar solvent in an amount less than 5% w/w of the formulation.
30. The transdermal delivery formulation of claim 1, wherein the transdermal delivery formulation further comprises a detergent portion in an amount between about 1-30% w/w of the transdermal delivery formulation.
31. The transdermal delivery formulation of claim 30, wherein the detergent portion comprises a nonionic surfactant in an amount between about 2-25% w/w of the transdermal delivery formulation; and a polar solvent in an amount less than 5% w/w of the transdermal delivery formulation.
32. The transdermal delivery formulation of claim 1, wherein the transdermal delivery formulation is in an amount between about 10-60% w/w of the transdermal delivery formulation.
33. The transdermal delivery formulation of claim 1, wherein the transdermal delivery formulation comprises an alcohol in an amount less than 10% w/w of the transdermal delivery formulation.
34. The transdermal delivery formulation of claim 1, wherein the transdermal delivery formulation further comprises an alcohol, a surfactant, and a polar solvent.
35. The transdermal delivery formulation of claim 1, wherein the transdermal delivery formulation comprises cetyl alcohol in amount less than 5% w/w of the formulation.
36. The transdermal delivery formulation of claim 1 wherein the transdermal delivery formulation comprises ethanol in an amount less than 5% w/w of the formulation.
37. The transdermal delivery formulation of claim 1 wherein the transdermal delivery formulation comprises glycerine in an amount less than 5% w/w of the formulation.
38. The transdermal delivery formulation of claim 1 wherein the transdermal delivery formulation comprises propylene glycol in an amount less than 8% w/w of the formulation.
39. The transdermal delivery formulation of claim 1, wherein the formulation comprises a gelling agent in an amount less than 20% w/w of the formulation.
40. The transdermal delivery formulation of claim 1, wherein the formulation comprises menthol in an amount between about 0.05-5% w/w of the formulation.
41. The transdermal delivery formulation of claim 1, further comprising tranexamic acid in an amount less than 5% w/w of the formulation.
42. The transdermal delivery formulation of claim 1, further comprises a humectant, an emulsifier, an emollient, or a combination thereof.
43. The transdermal delivery formulation of claim 1, wherein the formulation has a pH of 9-11.
44. The transdermal delivery formulation of claim 1, wherein the formulation has a pH of 7-10.5.
45. The transdermal delivery formulation of claim 1, further comprising an active agent.
46. The transdermal delivery formulation of claim 1, further comprising an active agent component in an amount less than about 60% w/w.
47. A transdermal delivery formulation to effect transdermal delivery of an active ingredient comprising applying to the skin, nails or hair follicles of a subject an effective amount of the formulation of claim 46.
48. The transdermal delivery formulation of claim 1, wherein the formulation includes an active agent to treat a disease.
49. The transdermal delivery formulation, wherein the disease is a cancer, a kidney disease, gout, melasma, a heart condition or a dermal disease.
50. The transdermal delivery formulation of claim 1, wherein the formulation has a pH of 4.5-10.5.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A transdermal delivery formulation for transdermal delivery of an active agent through the skin, nail or hair follicle of a subject, wherein the transdermal delivery formulation comprises:
a) a transdermal delivery formulation in an amount less than about 60% w/w, comprising:
iv. one or more phosphatides,
v. glucose, and
vi. one or more fatty acids; and
b) water in an amount less than about 50% w/w.

2. The transdermal delivery formulation of claim 1, further comprising benzyl alcohol in an amount between about 0.5-5% w/w.

3. The transdermal delivery formulation of claim 1 wherein the transdermal delivery formulation comprises benzyl alcohol in an amount less than 5% w/w of the formulation.

4. The transdermal delivery formulation of claim 1, further comprising Isopropyl Palmitate in an amount between about 5-5% w/w.

5. The transdermal delivery formulation of claim 1, wherein the water is deionized water and/or purified water.

6. The transdermal delivery formulation of claim 1, wherein the water is not deionized water and/or purified water.

7. The transdermal delivery formulation of claim 1, wherein the water is in an amount between about 15-40% w/w of the formulation.

8. The formulation of claim 1, wherein the one or more phosphatides in an amount between about 0.5-55% w/w of the transdermal delivery formulation.

9. The transdermal delivery formulation of claim 1, wherein the transdermal delivery formulation comprises phosphatidylcholine, hydrogenated phosphatidylcholine, phosphatidylserine, phosphatidylethanolamine, phosphatidylinositol, or a combination thereof in amount less than 30% w/w of the formulation.

10. The transdermal delivery formulation of claim 1, wherein the one or more phosphatides comprises phosphatidylcholine of the transdermal delivery formulation.

11. The transdermal delivery formulation of claim 1, wherein the one or more fatty acids in an amount between about 1-35% w/w of the transdermal delivery formulation.

12. The transdermal delivery formulation of claim 1, wherein the one or more fatty acids in an amount between about 5-35% w/w of the transdermal delivery formulation.

13. The transdermal delivery formulation of claim 1, wherein the one or more fatty acids comprises Linoleic Acid, Oleic Acid, Stearic Acid, safflower oil, or a combination thereof.

14. The transdermal delivery formulation of claim 1, wherein the one or more fatty acids comprises Linoleic Acid.

15. The transdermal delivery formulation of claim 1, wherein the one or more fatty acids comprises Oleic Acid.

16. The transdermal delivery formulation of claim 1, wherein the one or more fatty acids comprises Stearic Acid.

17. The transdermal delivery formulation of claim 1, wherein the one or more fatty acids are derived from a seed oil in an amount between about 0.5-55% w/w of the transdermal delivery formulation.

18. The transdermal delivery formulation of claim 1, wherein the one or more fatty acids are derived from a seed oil in an amount between about 5-35% w/w of the transdermal delivery formulation.

19. The transdermal delivery formulation of claim 1, wherein the one or more fatty acids are derived from a safflower oil in an amount between about 0.5-55% w/w of the transdermal delivery formulation.

20. The transdermal delivery formulation of claim 1, wherein the one or more fatty acids are derived from a safflower oil in an amount between about 5-35% w/w of the transdermal delivery formulation.

21. The transdermal delivery formulation of claim 1, wherein the one or more fatty acids are derived from an almond oil in an amount between about 0.5-55% w/w of the transdermal delivery formulation.

22. The transdermal delivery formulation of claim 1, wherein the one or more fatty acids are derived from an almond oil in an amount between about 5-35% w/w of the transdermal delivery formulation.

23. The transdermal delivery formulation of claim 1, wherein the one or more fatty acids comprises one or more fatty acids derived from soy lecithin.

24. The transdermal delivery formulation of claim 1, wherein the glucose in an amount between about 0.05-10% w/w of the transdermal delivery formulation.

25. The transdermal delivery formulation of claim 1, wherein the glucose is anhydrous dextrose in an amount between about 0.05-10% w/w of the transdermal delivery formulation.

26. The transdermal delivery formulation of claim 1, further comprises a nonionic surfactant in an amount between about 2-25% w/w of the transdermal delivery formulation.

27. The transdermal delivery formulation of claim 26, further comprises a polar solvent at least in an amount in molar excess of the nonionic surfactant.

28. The transdermal delivery formulation of claim 27, wherein the nonionic surfactant is a poloxamer and the polar solvent is water.

29. The transdermal delivery formulation of claim 1, further comprising a polar solvent in an amount less than 5% w/w of the formulation.

30. The transdermal delivery formulation of claim 1, wherein the transdermal delivery formulation further comprises a detergent portion in an amount between about 1-30% w/w of the transdermal delivery formulation.

31. The transdermal delivery formulation of claim 30, wherein the detergent portion comprises a nonionic surfactant in an amount between about 2-25% w/w of the transdermal delivery formulation; and a polar solvent in an amount less than 5% w/w of the transdermal delivery formulation.

32. The transdermal delivery formulation of claim 1, wherein the transdermal delivery formulation is in an amount between about 10-60% w/w of the transdermal delivery formulation.

33. The transdermal delivery formulation of claim 1, wherein the transdermal delivery formulation comprises an alcohol in an amount less than 10% w/w of the transdermal delivery formulation.

34. The transdermal delivery formulation of claim 1, wherein the transdermal delivery formulation further comprises an alcohol, a surfactant, and a polar solvent.

35. The transdermal delivery formulation of claim 1, wherein the transdermal delivery formulation comprises cetyl alcohol in amount less than 5% w/w of the formulation.

36. The transdermal delivery formulation of claim 1 wherein the transdermal delivery formulation comprises ethanol in an amount less than 5% w/w of the formulation.

37. The transdermal delivery formulation of claim 1 wherein the transdermal delivery formulation comprises glycerine in an amount less than 5% w/w of the formulation.

38. The transdermal delivery formulation of claim 1 wherein the transdermal delivery formulation comprises propylene glycol in an amount less than 8% w/w of the formulation.

39. The transdermal delivery formulation of claim 1, wherein the formulation comprises a gelling agent in an amount less than 20% w/w of the formulation.

40. The transdermal delivery formulation of claim 1, wherein the formulation comprises menthol in an amount between about 0.05-5% w/w of the formulation.

41. The transdermal delivery formulation of claim 1, further comprising tranexamic acid in an amount less than 5% w/w of the formulation.

42. The transdermal delivery formulation of claim 1, further comprises a humectant, an emulsifier, an emollient, or a combination thereof.

43. The transdermal delivery formulation of claim 1, wherein the formulation has a pH of 9-11.

44. The transdermal delivery formulation of claim 1, wherein the formulation has a pH of 7-10.5.

45. The transdermal delivery formulation of claim 1, further comprising an active agent.

46. The transdermal delivery formulation of claim 1, further comprising an active agent component in an amount less than about 60% w/w.

47. A transdermal delivery formulation to effect transdermal delivery of an active ingredient comprising applying to the skin, nails or hair follicles of a subject an effective amount of the formulation of claim 46.

48. The transdermal delivery formulation of claim 1, wherein the formulation includes an active agent to treat a disease.

49. The transdermal delivery formulation, wherein the disease is a cancer, a kidney disease, gout, melasma, a heart condition or a dermal disease.

50. The transdermal delivery formulation of claim 1, wherein the formulation has a pH of 4.5-10.5.
